(19) United States
(12) Patent Application Publication
(10) Pub. No.: US 2025/0127634 A1
(43) Pub. Date: Apr. 24, 2025

(54) PROSTHETIC FOOT WITH SPACED SPRING ELEMENTS

(71) Applicant: OTTO BOCK HEALTHCARE LP, Austin, TX (US)

(72) Inventors: Jesse DAY, Holladay, UT (US); Vijay MORAMPUDI, Midvale, UT (US); Brandon ANDERSON, West Jordan, UT (US); Joanna SWEETGALL, Salt Lake City, UT (US); Randy HALL, Riverton, UT (US); Darin RAY, Centerville, UT (US); Carsten MOENICKE, Duderstadt (DE)

(21) Appl. No.: 18/979,306

(22) Filed: Dec. 12, 2024

Related U.S. Application Data
(63) Continuation of application No. 16/225,459, filed on Dec. 19, 2018.
Application No. 16/225,459 is a continuation-in-part of application No. 15/305,633, filed on Oct. 20, 2016, now Pat. No. 10,881,533.
(60) Provisional application No. 62/664,538, filed on Apr. 30, 2018.

(30) Foreign Application Priority Data
May 7, 2014 (DE) 102014006571.5

(51) Int. Cl. A61F 2/66; A61F 2/50; A61F 2/76

(52) U.S. Cl. CPC A61F 2/66; A61F 2/6607; A61F 2/76; A61F 2002/5001; A61F 2002/5003; A61F 2002/5007; A61F 2002/503; A61F 2002/6621; A61F 2002/6642; A61F 2002/665; A61F 2002/6657; A61F 2002/6664; A61F 2002/6671; A61F 2002/6678

(57) ABSTRACT

A prosthetic foot includes a base spring, a top spring assembly, a connector assembly, and a heel cushion. The top spring assembly includes first and second spring members, and first and second bond connections. The second spring member extends parallel with and spaced apart from the first spring member. The first bond connection is between distal ends of the first and second spring members, and the second bond connection is between a distal end of the second spring member and a top surface of the base spring. The top spring assembly has a first portion arranged horizontally, and a second portion arranged vertically. The connector is connected to a proximal end of the top spring assembly to connect the prosthetic foot to a lower limb prosthesis. The heel cushion is mounted to the base spring at a location spaced forward of a heel end of the base spring.

### CROSS REFERENCE TO RELATED APPLICATIONS

This application is a continuation of U.S. patent application Ser. No. 16/225,459, filed 19 Dec. 2018, and entitled PROSTHETIC FOOT WITH SPACED SPRING ELEMENTS, which is a continuation-in-part of U.S. patent application Ser. No. 15/305,633, filed 20 Oct. 2016, and entitled PROSTHETIC FOOT, pending, now U.S. Pat. No. 10,881,533, which is a U.S. National Entry Application from PCT International Patent Application No. PCT/EP2015/000926, filed 6 May 2015, and also entitled PROSTHETIC FOOT, which claims benefit of German Patent Application No. 102014006571.5, filed 7 May 2014, the disclosures of which are incorporated, in their entireties, by this reference. U.S. patent application Ser. No. 15/305,633 also claims the benefit of U.S. Provisional Patent Application No. 62/664,538, filed 30 Apr. 2018, and entitled PROSTHETIC FOOT WITH SPACED ELEMENTS. The disclosures of the above applications are incorporated, in their entirety, by this reference.

### TECHNICAL FIELD

The present disclosure relates generally to prosthetic devices, and more particularly relates to prosthetic feet having improved force absorption. The disclosure also relates to a prosthetic foot comprising a structural component with proximal connection features for fastening the prosthetic foot to a below knee tube, below knee shank or a prosthetic knee joint, a forefoot portion fastened to, or embodied at, the structural component and a heel-side spring-damper system assigned to the structural component, the spring-damper system being compressed at a heel strike and supporting itself on a sole-side guide element. The prosthetic foot may also be suitable for arrangement in a shoe and embodied to this end.

### BACKGROUND

Prosthetic feet serve as distal termination for a prosthetic device and may be fixed to a below knee tube, which is fastened to a prosthetic knee joint, directly to a prosthetic shank or to the prosthetic knee joint. To this end, connection features are regularly provided at the proximal end on the prosthetic foot in order to establish a stable and permanent connection with the proximal prosthetic component. Prosthetic feet are usually provided with a cosmetic covering, which consist of plastic and are embodied approximately in the form of a natural foot.

From the structural point of view, the simplest form of a prosthetic foot is a rigid foot. However, a rigid foot has significant disadvantages in view of the elastic properties or the rollover properties. More complex designs include dampening elements or heel springs for damping the momentum upon heel strike. It is likewise possible for a spring to be arranged in the forefoot region in order to enhance the rollover characteristics of the foot during the stance phase and to store and then release deformation energy so as to assist the prosthetic foot user when walking.

Prosthetic feet also serve as distal termination for a prosthetic device and may be fixed to a below knee tube, which is fastened to a prosthetic knee joint, directly to a prosthetic shank or to the prosthetic knee joint. To this end, connection features are regularly provided at the proximal end on the prosthetic foot in order to establish a stable and permanent connection with the proximal prosthetic component. Prosthetic feet are usually provided with cosmetic features, which consist of plastic and are embodied approximately in the form of a natural foot.

Damper elements or heel springs may be provided for damping the momentum upon heel strike; it is likewise possible for a spring to be arranged in the forefoot region in order to ease the rollover of the foot over the whole stance phase and, moreover, to re-emit the deformation energy, previously taken-in, in the terminal stance phase so as to assist the prosthetic foot user when walking.

U.S. Pat. No. 7,172,630 relates to a prosthetic foot comprising two leaf spring elements which are coupled to one another in the forefoot region. A cam which presses against a tension spring is arranged at one leaf spring. It is possible to tension or relax the tension spring by displacing the cam such that a specific force profile may be realized over the heel-toe movement.

U.S. Pat. No. 5,139,525 relates to a prosthetic foot with an articulated receptacle for a below knee tube. The articulated receptacle is arranged in the region of the natural ankle joint. The spring characteristic of the prosthetic foot can change over the course of a heel-toe movement.

U.S. Patent Publication No. 2007/0061016 A1 relates to a prosthetic foot with a heel plate and a toe plate which are connected to one another in an articulated manner, swivelable about a central axis. A spring is arranged between the heel plate and the toe plate and supports itself at a rearward extension of the toe plate. An adapter plate is likewise arranged in a manner swivelable about the central axis. The energy arising when walking is actively stored and re-emitted by way of sensor devices.

U.S. Pat. No. 6,719,807 relates to a prosthetic foot comprising a wavelike-contoured forefoot spring and a heel spring, which is fastened in the midfoot region, extends backward and is arranged in a cosmetic foot covering. A forefoot spring and a heel spring are mounted in a frame. A variant provides for the heel spring to be connected to the forefoot spring by way of a base spring which is fastened, firstly, to the posterior end of the heel spring and, secondly, to the forefoot spring in the midfoot region.

U.S. Patent Publication No. 2012/0046760 relates to a prosthetic foot comprising an integral spring which comprises a lower base portion and an upper part rising upward in an arcuate manner. The base portion and upper part are provided with a longitudinal slot in the forefoot region; a damper made of an elastomeric material is arranged in the heel region.

U.S. Patent Publication No. 2014/0046456 relates to a prosthetic foot comprising a planar base spring, an arcuate forefoot spring with a connection adapter fastened thereto and a damper element which supports the base spring against the forefoot spring in the region of the heel.

A prosthetic foot for amateur athletes and runners is distributed by Chas A Blatchford & Sons Ltd. under the trade name “endolite Blade XT”; it comprises a forefoot spring comprising a substantially horizontal head portion and an integral spring with an outwardly convex form, said spring being divided into two in the lower region by a slit. In the front region of the spring, a sole protection and a heel spring are fastened by way of two screws; the spring stiffness of the heel spring may be set by way of a wedge. The heel spring has a less flexible reaction as a consequence of inserting the heel wedge.

Opportunities exist for improvements in prosthetic feet designs to improve energy feedback, dampening, and other properties that increase performance and user comfort and stability.

### SUMMARY

The present disclosure relates to a prosthetic foot which has a simple design, simplifies walking for a prosthetic foot user and, in particular, is advantageously usable in the case of sports activities. According to one aspect of the present disclosure, this object is achieved by a prosthetic foot having a structural component with proximal connection features for fastening the prosthetic foot to a below knee tube, a below knee shank or a prosthetic knee joint, a forefoot portion fastened to, or embodied at, the structural component and a heel-side spring-damper system assigned to the structural component, the spring-damper system being compressed at a heel strike and supporting itself on a sole-side guide element, provides for the structural component to be embodied as a leaf spring, which extends from the proximal connection features in the posterior direction, forms an arc and is guided in the anterior and distal directions, with the arc projecting beyond the guide element in the posterior direction. As a result of the heel-side spring-damper system, which is connected to the support structure by way of the guide element, it is possible to enable guidance of the displacement movement of the spring-damper system and of the guide element assigned to the spring-damper system when the heel is loaded. As a result, a pronation and supination movement during impact is at least reduced, as a result of which more precise guidance of the prosthetic foot may be realized during walking. Moreover, there is a transfer of the energy stored in the spring-damper system to the structural component during the rollover from the heel to the forefoot by virtue of the spring-damper system relaxing, releasing energy and, as a result, bringing about a delay of a deformation of the structural component. As a result of embodying the structural component as a leaf spring, it is possible to store the energy taken up during walking, in particular during running, and provide a long deformation path such that a large energy storage capability during the stance phase may be provided. As a result of the arc projecting beyond the guide element toward the rear, there is significant lengthening of the effective spring length, and so a high degree of deformation and deformability of the structural component in the form of the leaf spring is provided. Moreover, the arc renders it possible to obtain a low prosthetic foot height despite a long effective spring length and, at the same time, the wearability in the shoe is improved as a result of the rearward projection in the region of the natural ankle joint.

The arc of the structural component may project beyond the natural ankle position in the posterior direction and is arranged approximately level with the natural ankle position, or going slightly therebeyond, such that the apex of the arc lies behind the posterior, rear end of the guide element. The guide element is advantageously arranged at the lower side of the spring-damper system and, as a leaf spring in the forefoot portion of the structural component, fixedly mounted thereon. Here, the guide element is advantageously embodied to be thinner than the leaf spring of the structural component with the forefoot portion, can optionally be embodied as leaf spring or integrally formed on the structural component, in order to reduce pretension of the forefoot portion during a heel strike where possible. As a result of the relatively thin embodiment of the guide element, a greater deformability during a heel strike is ensured such that only a low force component and energy influx is introduced into the forefoot portion. The guide element may be mounted at the support structure with free movement or virtually free movement about an axis oriented transversely to the longitudinal extent of the prosthetic foot, for example by virtue of a type of hinge being embodied in the guide element. Here, an axis is not understood to mean a rail element but rather an imaginary line, at which components may be swiveled in relation to one another. By way of example, an axis is also present if a film hinge, an elastomeric element or a polycentric bearing is provided, by means of which two components are coupled to one another. For the purposes of forming a swivel axis, the guide element can have a film hinge which, firstly, enables folding-like swiveling about a horizontal axis but, secondly, prevents or reduces torsion about the longitudinal axis of the foot and the vertical axis of the foot.

The guide element may be borne directly at the structural component such that the axis, about which the guide element or parts of the guide element is/are freely movable or virtually freely movable, is set directly at the support structure or embodied by the guide element.

In addition to the embodiment of the guide element as a leaf spring, the former can also have a lug-like or folding-like embodiment, as a result of which an introduction of force into the forefoot portion is reduced further.

The spring-damper system may be advantageously embodied as a foam-material element or an elastomeric element, as a result of which it is possible to provide a multiplicity of forms, each having a different spring-damper characteristic, using cost-effective materials, and so it is easily possible to adapt the prosthetic foot to the desires or requirements of the prosthetic foot user. It is also possible to easily realize different heel heights. It is possible to set desired spring and damping properties in a cost-effective manner by way of a material mixture. As a result of embodying the spring-damper system as a foam-material element or elastomeric element, it is possible to build up only small shearing forces, or no shearing forces, and so no tensile forces or pressure forces are introduced into the guide element. Advantageously, the spring-damper element supports itself at the lower side of the structural component in the region of the natural ankle, advantageously in a region at which the arc starts such that there is support in the case of a support in the transition from the arc to the forefoot portion. This furthermore prevents there being an energy influx into the forefoot portion during the heel strike.

The spring-damper system may be reversibly arranged between the lower side of the support structure and the upper side of the guide element, for example by way of a holder which is fastened, preferably adhesively bonded, to the lower side of the structural component, into which holder the spring-damper system made of a foam-material element or an elastomeric element may be inserted with form fit. A form-fit element can likewise be arranged at the upper side of the guide element such that an adaptation to the desired stiffness or a replacement during servicing works can take place by simple insertion and latching of the spring-damper system.

The guide element is advantageously embodied as a lug or flap and may be embodied from a metal, in particular a light metal, or a plastics material. Here, the swivel axis, at which the guide element in the form of a lug or flap is mounted, adjoins the support structure such that a relatively long length may be realized for the guide element, as a result of which the spring-damper system may be set in a precise fashion.

A sole element made of an elastic material may be arranged at the lower side of the guide element, said sole element providing an additional spring and additional damping by way of the geometric dimensions and rigidity properties thereof such that the spring-damper system between the support structure and the guide element is not the only resilient element. As a result of the inherent elasticity of the sole element, it is possible to avoid and damp a sudden load uptake and load transfer onto the spring-damper system.

Arranged on the lower side of the guide element there may be a sole element made of an elastic material which, as a result of the geometric dimensions and stiffness properties thereof, is embodied in such a way that the center of pressure (COP) or point of attack of the force remains in the heel region for as long as possible during the rollover in order to avoid premature charging or a deformation of the forefoot spring.

The forefoot portion may be rigidly fastened to the structural component and it is advantageously embodied as a leaf spring or has at least one leaf spring or leaf-spring element. As a result of the rigid attachment to the support structure, it is possible to achieve precise guidance of the forefoot portion, as a result of which it is easier to control the prosthetic foot when walking. The embodiment of the forefoot portion as the spring renders it possible to take up energy when walking. Moreover, it is possible, after the strike with the forefoot portion after the heel strike, to transfer the stored energy from the spring-damper element to the forefoot, with the overall height of the prosthetic foot remaining constant, and so the force guidance is brought about via the floor or the sole structure. The energy stored in the spring-damper element is then emitted by way of the forefoot portion and eases the push off of the user at the end of the stance phase.

An overload stop may be arranged between the guide element and the support structure in order to prevent too great compression of the spring-damper system in the case of a massive energy influx, wherein an instability may arise during walking as a result of said too great compression.

The guide element may be mounted in a non-displaceable manner about an axis extending in the anterior-posterior direction and/or about an axis extending in the proximal-distal direction such that the guide element cannot carry out an avoidance movement and, in particular, supination and pronation of the guide element after the strike is prevented. The rigid mount about an axis extending in the anterior-posterior direction prevents the heel of the prosthetic foot bending away laterally when walking, which is advantageous, particularly when used as a foot for sports.

A heel in the proximal direction or convex arching may be embodied at the free end of the guide element in order to enable easy rollover and an adaptation of the sole structure, for example to the body weight or the gait of the patient.

The structural component itself can have an elastic embodiment, in particular an integral or single-piece embodiment such that a continuous leaf spring emerges from the proximal connection features up to the front end of the structural component, in particular up to the front end of the prosthetic foot.

A form-fit element for fixing the spring-damper system may be fixed in each case at the posterior end of the guide element and at the lower side of the structural component; in particular, the form-fit elements are adhesively bonded thereon. The form-fit element arranged at the guide element may be embodied as a heel cap and as a posterior, lower termination of the prosthetic foot and, at the same time, provide damping or cushioning in the heel region and a profile, to the extent that the prosthetic foot should not be worn in a shoe, such that risk of slipping prior to putting on the shoe is reduced. The upper form-fit element for form-fit locking to the lower side of the structural component can also be fixed or securely screwed thereon such that there may be simple assembly and the assembly of the spring-damper system, which can have an integral embodiment and consist of different materials.

The arc of the structural component projecting beyond the guide element toward the back projects beyond the spring-damper system in the anterior-posterior direction by a section which is between 10% and 30%, especially preferably between 12.5% and 25%, of the foot length. Here, the foot length is that length of the prosthetic foot which is measured from the front, anterior end to the rear, posterior end of the guide element, possibly to the rear end of the form-fit element arranged at the guide element.

Contour-forming cushioning may be arranged at the upper side of the structural component in the forefoot region, as a result of which cushioning it is possible to fill a shoe, into which the prosthetic foot is inserted, in a shape-forming manner. Hence, it is not necessary for further cosmetic coverings to be arranged around the prosthetic foot. Rather, the prosthetic foot according to the invention may be worn directly in a shoe.

In a development of the embodiments disclosed herein, provision may be made for a sole to be fastened to the prosthetic foot such that the latter can also be worn without a shoe.

From the proximal connection features, the structural component extends with concave curvature in the arc, convex curvature is present in the region of the support of the spring-damper system at the lower side of the structural component, said convex curvature advantageously having a larger radius of curvature compared to the arc. The convex region of curvature is in turn adjoined by concave curvature in the forefoot portion, wherein the perception of the structural component, proceeding from a top view in the region of the proximal connection features on the original surface, remains over the course from the upper connection features to the tip of the foot, i.e. initially looking from top to bottom and, after the end of the arc, looking from the bottom to the top.

The guide element may be embodied as a leaf spring with a form extending from the heel to the tip of the foot in a concave, convex, concave manner in order, firstly, to reproduce the natural arching of the foot and, secondly, to ensure soft rollover after the heel strike, high elasticity as a result of the convex movement in the midfoot region and soft strike and rollover in the forefoot region. Here, the perception is from the lower side of the guide element.

A development of the embodiments disclosure herein may provide for the forefoot portion and the guide element to be assigned to one another by way of an alignment device. The alignment device simultaneously provides a form for an adhesive connection and, moreover, protection for the components from external influences or for the shoe from possibly sharp-edged leaf springs since the alignment device is adhesively bonded together with the structural component and the guide element and remains at the prosthetic foot.

The forefoot portion, the guide element and the alignment device may be adhesively bonded to one another and, as a result thereof, permanently fastened to one another.

The prosthetic foot may be embodied and suitable for arrangement in a shoe, in particular as a result of the embodiment of the structural component with the rearward extended arc in the region of, or above, the natural ankle position and the guide element which assumes a sole function during the stance phase.

Another aspect of the present disclosure relates to a prosthetic foot having a base spring, a top spring assembly, a connector assembly, and a heel cushion. The base spring has a toe end portion and a heel end portion. The top spring assembly includes first and second spring members, first and second connections, first and second portions, and a slot. The first spring member has a distal end and a proximal end. The second spring member extends substantially parallel with and spaced apart from the first spring member along substantially an entire length of the first spring member. The second spring member has a distal end and a proximal end. The first connection is provided between the distal ends of the first and second spring members. The second connection is provided between the distal end of the second spring member and a top surface of the base spring. The first portion is arranged at a first angle relative to a horizontal plane. The second portion extends from the first portion. The slot extends rearward from the distal ends of the first and second spring members along at least half of a length of the first portion of the top spring assembly. The connector may be releasably connected to the proximal ends of the first and second spring members, and configured to connect the prosthetic foot to a lower limb prosthesis. The heel cushion is mounted to the base spring at a location spaced forward of a heel end of the base spring. The heel cushion is arranged to contact a bottom surface of the second spring member during use of the prosthetic foot.

The first angle of the top spring assembly may be in the range of about 5° to about 30°. The second portion of the top spring assembly may extend from the first portion in a substantially vertical direction. The prosthetic foot may further include a spacer positioned between the proximal ends of the first and second spring members. The first bond connection may provide a spacing between the distal ends of the first and second spring members. The first bond connection may include an adhesive. The second spring member may be spaced apart from the first spring member by spacers positioned at the distal and proximal ends of the first and second spring members. A thickness of the first spring member may be within 15% of a thickness of the second spring member.

Another aspect of the present disclosure relates to a prosthetic foot having a connector, a base spring, a top spring assembly, and a heel cushion. The connector is configured to connect the prosthetic foot to a lower limb prosthesis. The base spring has a toe end portion and a heel end portion. The top spring assembly includes a first spring member having a distal end and a proximal end, a second spring member positioned between the base spring and the first spring member, the second spring member having a distal end and a proximal end, and first and second spacers. The first spacer is positioned between the distal ends of the first and second spring members. The second spacer is positioned between the proximal ends of the first and second spring members. The first and second spacers are configured to space the second spring member away from the first spring member when the prosthetic foot is in a rest state. During use of the prosthetic foot, a portion of the first spring member spaced between the distal and proximal ends of the first spring member moves relative to the second spring member. The first spacer may be made of a wear resistant, low friction material allowing the distal end of the first spring member to slide relative to the second spring member in the anterior-posterior direction. The top spring assembly further includes a distal end portion connected to the base spring and extending substantially horizontally, and a proximal end portion connected to the connector. The heel cushion is mounted to the base spring at a location spaced forward of a heel end of the base spring. The heel cushion is arranged to contact a bottom surface of the second spring member during use of the prosthetic foot.

The distal end portion of the top spring assembly may be connected to the base spring with a permanent bond connection, and material of the permanent bond connection may include an elastomeric material. The proximal end portion of the top spring assembly may include the proximal ends of the first and second spring members and may be releasably connected to the connector with at least one fastener. The distal ends of the first and second spring members may be connected to each other with a bond connection, and material of the bond connection may define the first spacer. The top spring assembly may have a first portion arranged at an angle in the range of about 5° to about 30° relative to a horizontal plane, and a second portion extending substantially vertically. The top spring assembly may be spaced apart from the base spring. The material of the bond connection may include an elastomeric material. The heel cushion may be releasably attached to the base spring, and the heel cushion may include an elastomeric material. The distal end of the second spring member may extend distally further than the distal end of the first spring member.

The distal end portion of the top spring assembly may space apart the first and second springs with one or more blocks and/or pads of wear resistant, low friction material. The material may transfer compression forces between the first and second spring but allow the distal ends of the first and second springs to separate in the vertical direction and allow sliding between the distal ends in a horizontal direction. The material may also be selected to reduce noise when the first and second springs come into contact, such as during a gait cycle.

A further aspect of the present disclosure relates to a method of manufacturing a prosthetic foot. The method includes providing a base spring, a connector, a heel cushion, first and second spring members, and a spacer, arranging the first and second spring members in parallel with each other with portions of the second spring member positioned below and/or rearward of the first spring member, connecting distal ends of the first and second spring members to each other (e.g., by bonding using an elastomeric material), and arranging the spacer between proximal ends of the first and second spring members. The spacer spaces the first and second spring members apart when the prosthetic foot is in a rest position. Alternatively, both distal and proximal ends of the upper spring assembly may utilize spacers (e.g., spacers positioned where the proximal ends of the springs are fixed to each other, and where the distal ends slide against each other. The method also includes connecting the proximal ends of the first and second spring members to the connector with at least one fastener, connecting the distal end of the second spring member to a toe end portion of the base spring, the distal ends of the first and second spring members being arranged substantially parallel with a top surface of the base spring, and mounting the heel cushion to the base spring at a location spaced forward of a heel end of the base spring, the heel cushion contacting a bottom surface of the second spring member.

The proximal ends of the first and second spring members may be arranged substantially vertically. The method may also include providing the first and second spring members with a lower portion and an upper portion, arranging the lower portion at an angle of about 5° to about 30° relative to a horizontal plane, and arranging the upper portion substantially vertically. The base spring may include a sandal slot formed in the toe end portion, and bonding the distal end of the second spring member to the base spring may include connecting to the base spring at a location spaced posterior of the sandal slot. A portion of the first spring member may be movable into contact with the second spring member during use of the prosthetic foot. The connections between the first and second spring members, and between the top spring assembly and the base spring may be a bond connection using, for example, an elastomeric material. The material of the bond connections may space the first and second spring members from each other, and may space the top spring assembly from the base spring.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 shows a perspective view of a prosthetic foot.

FIG. 2 shows a side view of FIG. 1.

FIG. 3 shows a variant with a modified structural component.

FIG. 4 shows a variant of FIG. 3.

FIG. 5 shows a schematic illustration of a lateral-force compensation.

FIG. 6 shows a side view of an orthopedic component during manufacturing.

FIG. 7 shows a perspective view with feed and outlet devices.

FIG. 8 shows a sectional view of part of an orthopedic component.

FIG. 9 shows a perspective view of an alignment device.

FIG. 10 shows another view of the alignment device of FIG. 9.

FIG. 11 shows a partial illustration of an orthopedic component from obliquely behind.

FIG. 12 shows a variant of FIG. 11.

FIG. 13 shows a perspective partial illustration of a second embodiment.

FIG. 14 shows another view of the embodiment of FIG. 13.

FIG. 15 shows an overall view of an alignment device of the second embodiment.

FIG. 16 shows a sectional illustration of FIG. 15.

FIG. 17 shows a sectional illustration of FIG. 14.

FIG. 18 shows a different view of FIG. 17.

FIG. 19 is a front perspective view of an example prosthetic foot assembly in accordance with the present disclosure.

FIG. 20 is a rear perspective view of the prosthetic foot assembly of FIG. 19.

FIG. 21 is a front exploded perspective view of the prosthetic foot assembly of FIG. 19.

FIG. 22 is a rear exploded perspective view of the prosthetic foot assembly of FIG. 19.

FIG. 23 is a right side view of the prosthetic foot shown in FIG. 19.

FIG. 24 is a left side view of the prosthetic foot shown in FIG. 19.

FIG. 25 is a top view of the prosthetic foot shown in FIG. 19.

FIG. 26 is a bottom view of the prosthetic foot shown in FIG. 19.

FIG. 27 is a front view of the prosthetic foot shown in FIG. 19.

FIG. 28 is a rear view of the prosthetic foot shown in FIG. 19.

FIG. 29 is a cross-sectional view of the prosthetic foot shown in FIG. 25, taken along cross-section indicators 29-29.

FIG. 30 is a side view of another example prosthetic foot in accordance with the present disclosure.

FIG. 31 is a side view of another example prosthetic foot in accordance with the present disclosure.

FIG. 32 is a flow diagram illustrating an example method in accordance with the present disclosure.

FIG. 33 is a flow diagram illustrating another example method in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure is also generally directed to prosthetic devices, and more particularly relates to prosthetic foot devices, which are also referred to as a foot prostheses. The prosthetic foot embodiments disclosed herein may provide certain advantages as compared to other prosthetic foot devices. For example, the prosthetic foot devices of the present application may provide improved rollover characteristics, such as increased smoothness during rollover. The prosthetic foot may include an upper or top spring assembly having a pair of leaf springs attached at opposite ends with a gap provided between the leaf springs. The top spring assembly is combined with a base spring and is attached to the base spring at a toe region of the base spring. A heel cushion is positioned between the top spring assembly and the base spring in a heel region of the base spring. The heel cushion may be mounted directed to the base spring and arranged to contact a bottom surface of the top spring assembly. The heel cushion may be interchangeable to adjust heel stiffness.

The toe end of the prosthetic foot may include a split extending along a length dimension of the prosthetic foot. The split may be arranged along a longitudinal centered line of the prosthetic foot to provide medial/lateral compliance. The split may extend through portions of the base spring and the top spring assembly.

The prosthetic foot design is targeted generally to meet the needs of a typical, active amputee. The prosthetic foot is designed for comfortable walking and many common conditions encountered by an amputee, rather than being narrowly focused towards a particular user group such as runners, skiers, swimmers, etc.

The top spring assembly may have various shapes and designs. In one example, the top spring assembly includes a generally horizontal portion that extends substantially parallel to the base spring, and an upper portion that extends in a substantially vertical direction to provide an L-shaped construction. Other designs have a lower profile that does not include the vertical portion of the L-shaped design. Still further embodiments may include a vertical portion having a greater length that extends further vertically upward relative to a base spring.

The base spring may extend further in an anterior direction than the most anterior point of the top spring assembly. The base spring may include a sandal slot position adjacent to the split formed along the centerline that provides medial/lateral compliance. In at least some embodiments, the first and second leaf springs of the top spring assembly may terminate at different anterior locations (e.g., the upper most leaf spring being spaced further posteriorly as compared to the lower leaf spring). Further, a heel end of the base spring may extend further posteriorly beyond the heel cushion. This arranged at the heel end of the base spring provides a heel lever arm located posterior to the heel cushion.

The prosthetic foot of the present disclosure may include various combinations of features that provide certain advantages as compared to known prosthetic feet. Once such combination of features includes a multi-spring top spring assembly wherein the springs are connected at opposite ends and include a gap or spacing between each other along the lengths of the springs between the connection points at the opposite ends. The top spring assembly is connected to a full-length base spring in a toe region of the base spring, and at a location that is spaced posterior of an anterior end of the base spring. The top spring assembly may be connected to the base spring at only one location. The prosthetic foot includes a replaceable elastomeric heel cushion positioned between the base spring and the top spring assembly in a heel end of the prosthetic foot. Typically, the heel cushion is detached from the top spring assembly, but is arranged to contact a bottom surface of the top spring assembly, particularly during use. The top spring assembly may include leaf springs having different lengths. The different lengths may allow a gradual change in the stiffness of the top spring assembly at various locations along the length of the prosthetic foot (e.g., in the toe region). One advantage of this particular combination of features is that the flexibility of the base plate does not have as much influence on performance. To some degree, the base plate may, at the connection point to the top spring assembly, pivot or otherwise move due to the type of connection that is provided between the top spring assembly and the base spring. In at least one example, the connection is provided by an adhesive bond or other bond structure that provides at least some flexibility and/or elasticity. Heel performance of the prosthetic foot may also be enhanced by this pivoting provided by a flexible adhesive bond between the base spring and the top spring assembly, or may be accomplished by flexibility in the base spring. In this particular embodiment, the prosthetic foot includes bond flexibility at the connection between the base spring and the top spring assembly as well as flexibility in the base spring provided by the structure of the base spring and the split in the toe end portion. A flexible bond between the upper spring assembly and the distal plate may act as a film hinge as described in U.S. Patent Publication No. 2017/0049584, which is incorporated herein, in its entirety, by this reference. A flexible distal plate may also contribute to film hinge behavior. It may be possible to tailor the contribution of the distal plate to heel performance by adjusting these and other design parameters. The heel performance is primarily determined by compressibility of the heel cushion, although the shape and position of the heel cushion may also contribute to heel performance. Thus, various features contribute to achieving a desired heel function for the prosthetic foot. By transferring load during heel strike from the posterior portion of the base spring to the posterior portion of the top spring assembly, the heel cushion reduces stress at the connection between the base spring and the upper spring assembly located in the anterior region of the prosthetic foot.

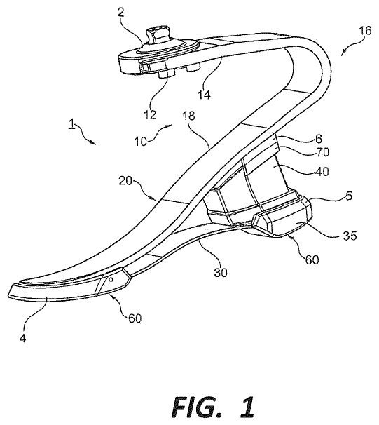

FIG. 1 shows, in a perspective side view, a prosthetic foot 1 comprising a structural component 10 in the form of an integral leaf spring, at the proximal end of which connection adapter 2 in the form of an adapter pylon are fastened by way of screws. Screwing is carried out in a support portion 14, which is directed out substantially horizontally and level in the case of a usual setting of the prosthetic foot 1. From the connection adapter 2, the structural component 10 extends rearward in an arc 16, which is oriented in the posterior direction, with convex curvature in order then to extend in the anterior direction in a connection portion 18 with a concave embodiment. The concave connection portion 18 is adjoined by a forefoot portion 20, once again with a convex shape, which extends into the toe region of the prosthetic foot 1. An alignment device 4 in the form of a cap is adhesively bonded to the toe region and has a sole element 60 at the lower side thereof, or at the lower side of which a sole element 60 is formed. The alignment device 4 serves, firstly, for receiving and fastening the forefoot portion 20 of the structural component 10, the protection thereof at the front end and at the side edges, and for an alignment in relation to a guide element 30, which is fixed below the forefoot portion 20, and at a distance thereto, at the alignment device. The manner of the fastening and the embodiment of the alignment device 4 are explained in more detail below.

The guide element 30 extends substantially horizontally in the posterior direction, with the guide element 30 being embodied as a leaf spring which, extending from front to rear, has a convex, concave and then convex contour profile again. Arranged at the posterior end of the guide element 30 is a form-fit element 5 in the form of a heel cap, at the lower side of which a sole element 60 is likewise arranged or formed. The form-fit element 5 can be fixed to the guide element 30 in a form-fit manner, for example by being plugged on, clipped on or screwed on; alternatively, or additionally, it is possible for adhesive bonding and/or welding to be carried out with the guide element 30.

Arranged at the lower side of the structural component 10 in the region of the connection portion 18 is a second form-fit element 6, which is advantageously adhesively bonded, welded or fastened thereon by form-fit elements such as screws or the like. Together with the first form-fit element 5, the second form-fit element 6 serves for reversible reception of a spring-damper system in the form of a foam-material body or of an elastomeric element which is used between the structural component 10 and the guide element 30. To this end, the guide element 30 is moved away from the structural component 10, the spring-damper system 40 is inserted into the increasing intermediate space between the lower side of the structural component 10 and the upper side of the guide element 30, pushed rearward and held, e.g. by way of projections and grooves, in a form-fit manner between the two resilient components of the prosthetic foot 10.

It is already possible to gather from FIG. 1 that the structural component 10 has an integral embodiment and is designed as a leaf spring, advantageously made of a fiber-reinforced polymer composite, and the arc 16, which has a form that is convexly arched toward the outside, projects beyond the rear end of the guide element 30 in the posterior direction. The spring-damper system 40 is dimensioned in such a way here that the part of the structural component 10 projecting beyond the rear end of the guide element 30 extends above a natural ankle, or in the region thereof, such that the prosthetic foot 1 can be readily inserted into a shoe.

As a result of the spring length that is increased in the posterior direction, it is possible to provide an increased overall spring length with, at the same time, a flat prosthetic foot such that a larger amount of energy can be stored and deeper sinking-in can occur during a heel strike without it being necessary for the material strength of the structural component 10 to be excessively increased, which would necessarily be to the detriment of the durability or adjustability and elasticity during a heel strike.

The guide element 30 is substantially thinner than the structural component 10 in order to ensure easy deformability when the heel strike occurs. As a result, the guide element 30 can easily be swiveled about an axis extending perpendicular to the running direction and extending in the plane of the guide element such that there is only a small energy transfer or low force introduction onto the forefoot region 20 via the guide element 30 in the case of a heel strike. Swiveling about a vertical axis or about an axis oriented along the longitudinal extent of the prosthetic foot 1 is not possible, or only possible to a very restricted extent, due to the structure of the guide element as a leaf spring, and so a supination movement or pronation movement of the guide element 30 is avoided when deflecting or compressing the spring-damper system.

The form-fit element 5, also referred to as heel cap, which is arranged at the rear end of the guide element 30 can form a heel such that there is increased clear space from the floor at the rear end of the guide element 30. This heel can be formed by the sole element 60, which may consist of a compressible, elastic material, so as to bring about a soft introduction of force into the guide element 30 in the case of a heel strike.

The forefoot portion 20 is embodied as a leaf spring and allows deformation under forefoot loading. The guide element 30 is attached to the structural component 10 in the sagittal plane with low moment levels. Here, the sole element 60 is embodied in such a way that, in addition to a compression of the spring-damper system 40, a displacement of the resultant floor reaction force fitting to the load sets in by virtue of loading in the heel region such that the point of force introduction migrates as uniformly as possible along the direction of longitudinal extent of the prosthetic foot 1 during the heel-toe movement. When rolling from the heel to the forefoot, there is a transfer of the energy stored after the initial heel strike in the spring-damper system 40 to the forefoot portion 20 by virtue of not only the forefoot spring being compressed but also the spring-damper system 40 being relaxed, releasing the energy stored therein and thereby reducing a delaying effect as a result of the deformation of the forefoot portion 20 of the forefoot.

An overload stop 70 is provided at the structural component 10, said overload stop 70 restricting the compression travel of the guide element 30, for example in the case of peak loads in extraordinary situations, e.g. in the case of a jump from an elevation or the like. The overload stop 70 is embodied as a projection of the upper form-fit element 6 such that, in the case of excessive load, the overload stop 70 can come into direct contact with the lower form-fit element 5 and prevents further deformation and compression of the spring-damper system 40.

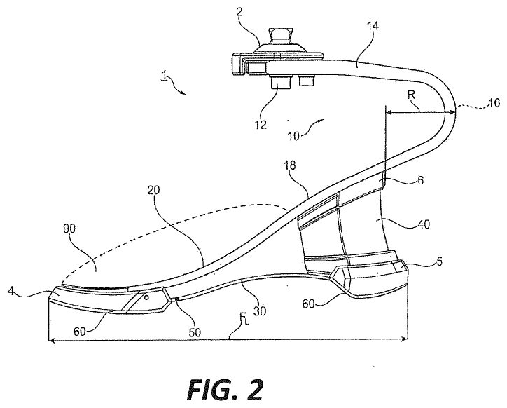

FIG. 2 shows the embodiment in accordance with FIG. 1 in a side view, comprising cushioning 90 which is arranged at the top side in the region of the forefoot portion 20 and which can be fixed reversibly to the top side, for example by way of a hook-and-loop fastener or the like. In the side view, it is possible to identify that the forefoot portion 20 extends up to the tip of the foot region of the prosthetic foot 1 and lies on the top side of the alignment device 4. The side edges of the structural component 10 are at least partly covered by sidewalls which project upwardly from the alignment device 4 such that protection of the sensitive side edges in the case of fiber-reinforced leaf springs is provided just like the protection for surrounding materials from the edges of the leaf spring. Moreover, the cushioning 90 can provide additional protection.

Within the alignment device 4, provision is made of an insertion opening (not depicted here), into which the guide element 30, which is likewise embodied in the form of a leaf spring, is inserted. The guide element 30 projects virtually completely into the alignment device 4; only a small tip region serves as a front termination and for protection against strike loads. The alignment device 4 is preferably made of an elastic material and adhesively bonded in a permanent manner to both the structural component 10 and the guide element 30. To this end, at least one cavity is arranged within the alignment device, said cavity being filled with an adhesive. A front sole element 60 can be arranged or formed at the lower side of the alignment device 4 in order to enable easy rolling over in a manner analogous to the sole element 60 in the case of the heel cap 5.

The heel cap 5 or the form-fit element 5 likewise has a reception slot for the guide element 30, the guide element 30 being inserted into the slot and held either by way of form-fit elements or by way of adhesive bonding or welding within the form-fit element 5.

In the side view, it is possible to identify that the guide element 30 has a form contoured from front to back with a form design that is initially convex, then concave and then convex again. As a result, the overall spring length is increased and a rollover movement is made easier, both during the heel strike and also during the terminal stance phase. The concave region of the guide element 30 is arranged in the midfoot region of the prosthetic foot 1 and models the natural arching of the foot.

Furthermore, it is possible to identify that the upper form-fit element 60 and hence also the upper contact face of the spring-damper system 40 with the connection portion at the lower side of the structural component 10 lie approximately in a plane with the rear end of the guide element 20. The arc 16 extending in the posterior direction beyond the form-fit element 6 and also beyond the posterior end of the guide element 30 has a projection R which is approximately one eighth of the overall foot length FL, i.e. the entire length from the tip of the foot to the posterior end of the heel cap 5. As a result of the backwardly oriented projection R, the effective spring length of the prosthetic foot 1 is massively increased, and so a greater energy storage capability can be achieved with, simultaneously, a greater compression travel and, at the same time, a narrow spring design since the stored energy is effected by way of the increased spring length and not by way of a thickening of the material in the leaf spring of the structural component 10. The thinner the design of a leaf spring made of a fiber composite is, the more durable it is since lower shearing forces occur within the leaf spring.

The contact face of the spring-damper system 40 with the structural component 10 is advantageously effected in the region of the connection portion 18, i.e. in the region in which the convex form of the arc 16 merges into the concave shape of the connection portion 18. The spring-damper system 40 made of a foam material or elastomeric element guides the forces occurring when treading during the initial heel strike directly in the direction of the connection adapter 2 due to the orientation of the spring-damper element in the direction of the connection adapter 2. Due to the introduction of the forces into the connection region, there is a compression during the heel strike over the proximally adjoining portion of the structural component 10, i.e. in the arc 16 and the connection portion 14, and so there is no force transmission and energy storage in the forefoot portion 20. That is to say, the forefoot portion 20 is not negatively charged during a heel strike since there is no introduction of force into the forefoot portion 20. Due to the low material strength of the guide element 30 and the simplified bending about a notional swivel axis 50 perpendicular to the plane of the sheet, there will also be no energy influx into the forefoot portion 20 by way of the guide element 30. When forming the spring-damper system 40 from a foam or corresponding elastomeric element, there are no shearing forces within the spring-damper element, and so no tensile forces are introduced into the guide element 30 either.

The spring-damper element 40, which may have an interchangeable design, advantageously has a progressive spring-damper behavior and can e.g. have a two-component foam, a two-component elastomer or a combination of a plurality of materials and/or a plurality of densities of the same materials in order to ensure the desired spring-damper properties. The spring-damper element 40 is held in a form-fit manner in the form-fit elements 5, 6; undercuts are provided in the form-fit elements 5, 6 which engage in recesses or grooves in the spring-damper system 40.

In the side view of FIG. 2, it is furthermore possible to identify that the structural component 10 is embodied as a single-piece leaf spring as a hinge-free prosthetic foot 1 and has a spring extension in the region of the ankle in order to increase the effective spring length. A vertical displacement of the prosthetic foot 1 is more easily possible as a result of the increased effective spring length, with the vertex of the arc 16 projecting beyond the natural overall foot length FL, i.e. beyond the rear end of the guide element 30 embodied as a base spring. The prosthetic foot 1 is immediately insertable into a shoe and does not require a cosmetic foot covering. On account of the design thereof, the prosthetic foot 1 has a high deformation capability, a large energy storage capacity during the stance phase, provides high durability due to the comparatively low material strength of the leaf spring component and enables a low prosthetic foot height. Moreover, the embodiment provides a heel component, and so it is possible to wear the prosthetic foot 1 during normal activities as well. Moreover, the prosthetic foot 1 is preferably suitable for sports activities such as jogging or the like. Sports feet known from the prior art can generally not be worn in a shoe and are generally not suitable for standing. Although conventional hinge-free prosthetic feet for daily use can be inserted into a cosmetic foot covering or possibly even directly into a shoe, they do not permit the high level of deformability afforded by the prosthetic foot 1 of the present invention.

In the side view in accordance with FIG. 2, the initially substantially horizontal leaf spring can be identified on the connection adapter 2 in the region of the connection portion 14, from which a slightly downwardly tilted course extends in the rearward, posterior direction. In principle, it is also possible for a further horizontal course or a slightly upwardly inclined course to join thereon. The connection portion 14 is adjoined by the rear arc 16, which is situated over approximately one eighth of the overall foot length FL behind the end of the heel of the prosthetic foot 1 and has concave curvature. At the end of the concave curvature, the structural component 10 merges into convex curvature with a larger radius compared to the arc 16 and from there it merges again into concave curvature with likewise a larger radius of curvature than the arc 16. During the heel strike, the spring-damper system 40 and the spring portion with the arc 16 and the connection portion 14 are connected in series, a deformation of the structural component 10 in the form of a displacement of the connection adapter 2 in the direction of the floor is possible as a result of the rear arc 16. To this end, the arc 16 projects over the line of the force which extends rearward from the heel strike to the connection adapter 2 through the spring-damper system. The greater the rearward projection R is, the softer the prosthetic foot 1 becomes; the maximum projection R emerges from the intended use, the employed materials and the preferences of the prosthetic foot user.

When rolling over to the mid-stance phase, the spring-damper system 40 is partly relieved since it is now only the forefoot portion 20 and the front sole element 60 which contact the floor. As a result, the whole structural component 10 begins to deform. The previously still non-deformed forefoot portion 20 is bent, the vertical force introduced during the mid-stance phase is converted into potential energy by a further deformation in the region of the arc 16 and of the connection portion 14. As a result, the vertical strike load is reduced. In the case of further rolling over to the forefoot, the spring-damper system 40 is relieved completely and the structural component 10 carries the entire load. Due to the arc 16, this is initially primarily vertically instead of in the return when lifting the forefoot and turns in the direction of the running direction during the last third of unloading. The vertex of the arc lies approximately in the region of the natural ankle and is displaced over the natural ankle position in the posterior direction. The heel stiffness is primarily controlled by the spring-damper system, the arc 16 and the connection portion 14 of the structural component 10. The point of attack of the force is held close to the heel for as long as possible due to the shaping of the sole element 60 in order to design the deformation property of the heel to be comfortable for the user. As a result of the narrow embodiment of the guide element 30, little force is applied to the forefoot portion 20; the latter is reduced further by the twice curved shape of the heel element.

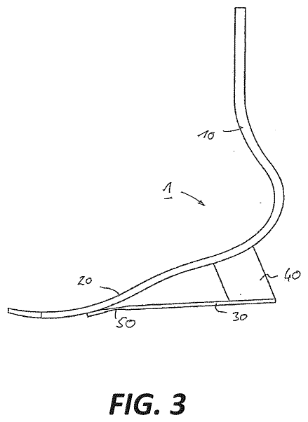

FIG. 3 shows a further variant of the prosthetic foot 1. In the depicted exemplary embodiment, the prosthetic foot 1 is equipped with an integral combination of the support structure 10 and the forefoot element 20. Then, there is fastening to a below knee shank or other fastening elements at the proximal end of the support structure 10. The plate-like, substantially planar guide element 30 is fastened, e.g. laminated, welded, screwed or adhesively bonded, to the floor-facing lower side in the ball region of the forefoot element 20. The axis 50, which is embodied as a swivel axis, is formed by a film hinge which allows swiveling about an axis lying within the film hinge; however, a rotation about a longitudinal axis in the anterior-posterior direction and about an axis in the proximal-distal direction is largely prevented due to the stiffness of the guide element 30. The spring-damper system is fastened immediately on the support structure 10 in the vicinity of the ankle.

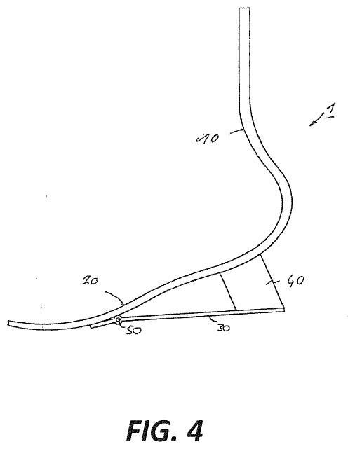

A variant of FIG. 3 is depicted in FIG. 4, in which a free hinge with an axis 50, which hinge can be embodied as a flap, is provided instead of a film hinge.

What is common to all embodiments is that a heel-side spring-damper system 40 is connected in the sagittal plane to further elements of the foot structure, i.e. either to the forefoot portion 20 or the structural component 10, with low moment levels by way of the guide element 30. Forces away from the effective direction of the spring-damper element 40 are taken up in the guide element 30 and dissipated by way of the hinge which permits swiveling about the axis 50. On the upper side thereof, the spring-damper system 40 is supported near the ankle at the structural component 10, the guide elements 30 being substantially embodied as planar structures and supporting the spring-damper system 40, which is supported at the lower side of the structural component, on the lower side. In the case of an eccentric force introduction into the heel, the planar structure of the guide element 30 can however twist such that the contact surface is enlarged. The horizontal forces, which are introduced when the heel is loaded and which act in the running direction or perpendicular to the running direction, are taken up by the structure of the guide element 30 and introduced into the support structure near the ankle by way of the hinge which is embodied as a swivel axis 50. Hence, the articulated fastening of the guide element 30 supports the heel-side spring-damper system against shearing in the case of horizontal forces. Here, the articulated mount is embodied in such a way that, as a result of the width thereof, it is well suited to take up the occurring shearing forces. It is also possible for a plurality of bearing points to be arranged next to one another on a common axis 50 in order to realize a successive arrangement of hinges.

Prosthetic feet are intended to soften the force during the treading strike, impart sufficient stability when rolling over and return that amount of energy during push off which the user can easily control when walking. The deformation of the individual components required to this end tests the capability of the employed high performance materials to their limits. Therefore, spring systems with elements which are connected to one another in a virtually rigid manner are often used in prosthetic feet made out of high performance materials. These elements protect one another from overload by the coupled effect but, on the other hand, do not allow independent effect on a different load introduction, e.g. from heel and forefoot.

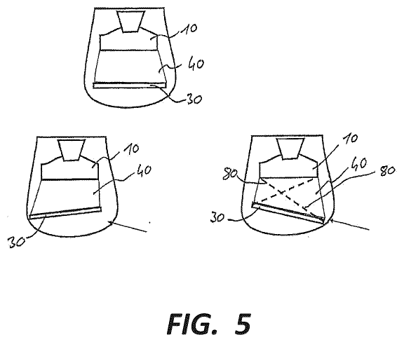

FIG. 5 shows, in a rear view, the structural component 10 and the spring-damper element 40, and also the guide element 30. Depicted in the upper illustration is an unloaded prosthetic foot; it is possible to identify that the guide element 30 is coupled to the structural component 10 by way of the heel-side spring-damper system 40. A force transfer from the guide element 30 to the structural component 10 should only take place within the effective direction of the spring-damper system 40; in the case of a heel strike, this is the direction of the force within the sagittal plane and from distal to proximal within the medial plane. Forces outside of the effective direction of the spring-damper system 40 are taken up by way of the guide element 30 and dissipated by virtue of the virtually articulated mount, either into the forefoot portion 20 and, via the latter, into the structural component 10 or directly into the structural component 10. The sole-side structure of the guide element 30 for guiding the spring-damper system 40 is embodied distally in such a way that, as a result of loading the heel and in addition to a compression of the spring-damper system 40, a displacement of the resultant floor reaction force, which fits to the loading, sets in, for example as a result of the torsion of the guide element 30. Such a situation is shown in the left-hand lower illustration of FIG. 5. A lateral force acts on the guide element 30, leading to displacement of said guide element 30.

In the right-hand lower illustration of FIG. 5, provision is made of an exemplary embodiment with two stabilizing elements 80 in the form of tension elements which are arranged in a crossing manner. Here, the guide element 30 is mounted and supported by the stabilizing elements 80 in such a way that a lateral force is not only supported but that the laterally attacking force is counteracted on the side of the force effect by a swivel movement by way of a displacement of the point of attack of the force.

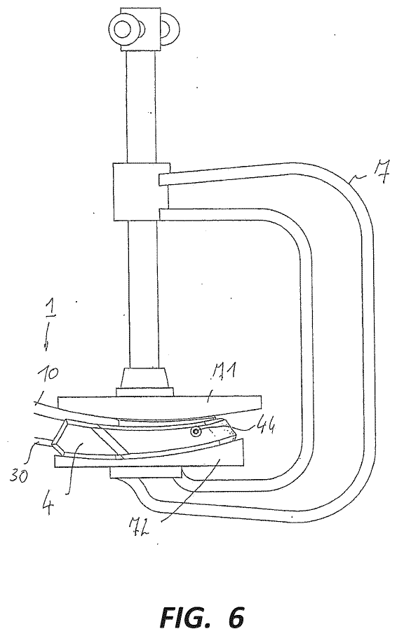

In a side view, FIG. 6 shows a schematic illustration of a front part of a prosthetic foot 1. The prosthetic foot 1 has two structural components 10, 30 which are produced as leaf springs from a fiber-reinforced plastic. The forefoot region of the prosthetic foot 1 is depicted; the first structural component 10 is a forefoot spring, the second structural component 30 is the guide element. The forefoot spring 10 extends obliquely upward to an upper connection point, at which fastening devices or connection features for fastening to a below knee tube or a below knee shank are able to be fastened. The guide element 30, which is also referred to as a base spring, leads into the heel region, wherein a heel spring can extend from the base spring 30 to the forefoot spring 10 and/or to the upper connection feature.

The structural component 10 and the guide element 30 are assigned to an alignment device 4 which is embodied as a plastic molded part. The alignment device 4 can consist of a polyurethane, a technical polyethylene, a technical polyurethane, rubber or any other plastic, preferably an elastomer. The alignment device 4 has an insertion slot for the guide element 30 and a reception region at the upper side for the first structural component 10, onto which the first structural component 10 can be supported. The support region is framed by walls such that the first structural component 10 can be supported with a defined position in relation to the alignment device 4 when the contour of the structural component 10 rests against the walls around the support region.

The guide element 30 is inserted into a slot (not depicted here) within the alignment device such that the lower side of the guide element 30 or of the leaf spring is covered by a closed surface of the lower side of the alignment device 4. A spacer is formed between the structural component 10 and the guide element 30, said spacer holding the two components securely at a distance from one another. As a result of inserting the guide element 30 into the alignment device 4, said former component is also assigned in a defined manner, for example by virtue of being guided in a slot or in a groove within the receiving device 4. As a result, the two components 10, 30 and the receiving device 4 form a cavity which is substantially closed-off. A feed connection 44 is provided in a side wall of the alignment device 4, said feed connection having a fluidic connection with the cavity (not depicted here), through which it is possible to insert or pump adhesive into the cavity. On the side distant from the feed connection 44, provision is made of an outlet channel, which likewise has a fluidic connection to the cavity, such that the air situated within the cavity can emerge and the cavity can be completely filled with adhesive.

The components 10, 30 and the alignment device 4 are held in a press 7, which can be formed as a conventional vice. Two press shoes 71, 72 are arranged on the press 7 and have a contour corresponding to the respectively assigned contour of the prosthetic foot 1. In the illustrated exemplary embodiment the upper press shoe 71 is provided with a convex curvature and the lower press shoe 72 is provided with a concave curvature so that on the one hand the lower side of the receiving device 4 and on the other hand the upper side of the first structural component 10 can bear over the entire surface against the surface of the relevant press shoe 71, 72. If the press 7 is closed and pressure is exerted onto the press shoes 71, 72, the first structural component 10 will be pressed against the surface of the support face on the alignment device 4 so that the cavity formed between the structural component 10 and the guide element 30 above and below and at the side faces by means of the alignment device 4 is closed and adhesive can be fed only through the feed connection 44, and air and any excess adhesive can escape through the outlet channel.

Following the introduction of the adhesive, the pressing force is maintained until the adhesive has cured, so that a permanent connection between the first structural component 10, the guide element 30, and the alignment device 4 is obtained. After curing of the adhesive, the alignment device 4 remains on the prosthetic foot 1 and serves in turn as protection for the structural component 10 and the guide element 30 and, secondly, as functional component of the orthopedic component, for example as a shaping for the prosthetic foot, as a cushion, as a sole structure, or in other embodiments as a receiving device or protective device for further components.

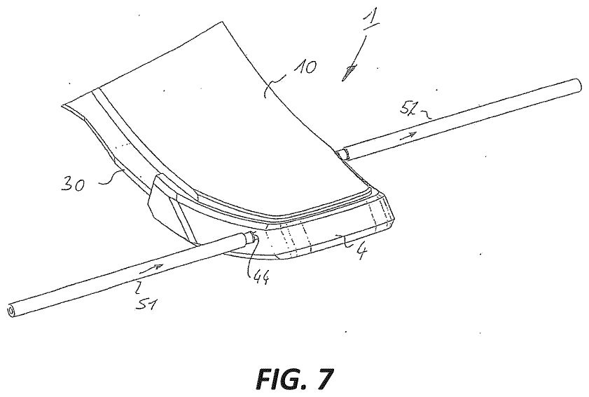

FIG. 7 shows, in a perspective oblique plan view, the manufacture of the prosthetic foot 1, or at least the connection of the structural component 10 and the guide element 30 to the alignment device 4. A feed device 51 is attached to the alignment device 4 at the feed connection 44, which feed device in the illustrated exemplary embodiment is formed as a tube or pipe and through which adhesive is introduced into the cavity (not illustrated) as indicated by the arrow. The cavity is formed and closed on the upper side and on the lower side by the structural component 10 and the guide element 30, on the front side and on the side edges by the side walls of the alignment device 4, and on the rear side between the leaf springs 10, 30 by a spacer, which bears tightly both against the lower side of the first structural component 10 and against the upper side of the guide element 30. The press 7 is not illustrated in FIG. 7; however, the assignment of the respective components 4, 10, 30 by the press 7 or another suitable fixing device is maintained during the feed of the adhesive.

Adhesive is introduced into the cavity through the feed device 51 and the feed connection 44, and the air disposed in the cavity is displaced by the adhesive and is transported away by an outlet device 52. The outlet device 52 is connected at an outlet channel (not illustrated), which is fluidically connected to the cavity within the receiving device 4, so that air and any excess adhesive can escape from the outlet channel through the outlet opening 51, as indicated by the arrow. Both the feed connection 44 and the outlet channel are preferably arranged in a spacer, which ensures that the leaf springs 10, 30 are held at a distance from one another. It is thus ensured that, by the arrangement of the leaf springs 10, 30 on or in the alignment device 4, are not blocked in relation to one another by the assignment of the leaf springs 10, 30.

The press 7 (not illustrated) holds the assignment of the components 4, 10, 20 in relation to one another until the adhesive has cured. Once the adhesive has cured, the feed device 51 and the outlet device 52 are separated from the alignment device 4, for example snapped off, so that a practically smooth termination of the alignment device 4 in the region of the feed connection 44 and the outlet channel can be achieved. This can be ensured for example by a predetermined breaking point on the feed device 51 and/or the outlet device 52 in the region of the connection to the alignment device 4.

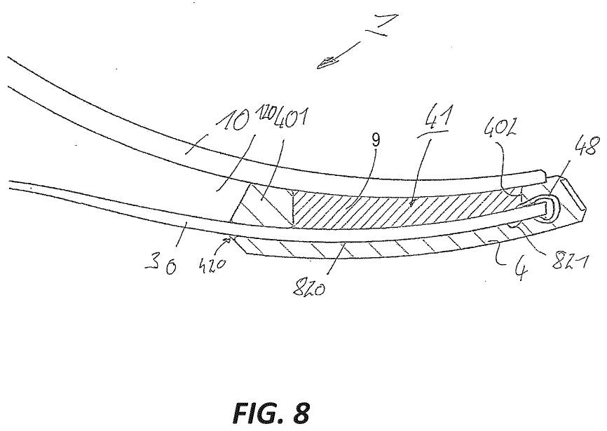

FIG. 8 shows a sectional illustration through the front part of a finished, assembled prosthetic foot 1 with an upper first structural component 10 resting on the alignment device 4, said structural component 10 being in the form of a forefoot spring made of a fiber-reinforced plastics material, with the alignment device 4, and with the guide element 30 inserted into the alignment device 4, said guide element being in the form of a base spring, which is likewise formed as a leaf spring made of a fiber-reinforced plastics material. The upper leaf spring rests on an upper support face, and the lower leaf spring rests on a lower support face 820. A channel 48 is formed at the front end (on the right-hand side in the illustrated exemplary embodiment) of the alignment device 4 and leads from the lower side of the guide element 30 to the cavity 41, which is enclosed by the guide element 30, the first structural component 10, and the alignment device 4. Indentations 821 are formed in the support face 820, which is formed by the surface of the base of the alignment device 4 facing toward the guide element 30, so that adhesive 9 completely filling the cavity 41 can infiltrate the indentations 821 also below the guide element 30 on account of a structured surface or the indentations 821, which are fluidically connected to the cavity 41, such that at least the lower guide element 30 is surrounded by a number of sides or at a number of points by the adhesive 9. A feed connection 44 is advantageously arranged at the geodetically lowest point of the alignment device 4 during the assembly, for example on the lower side of the alignment device 4 in the case of the presented orientation, and is fluidically connected both to the indentations 821 and, on account of the channel 48, also to the cavity 41. If adhesive 9 is now fed at the lowest point, said adhesive pushes through the structured surface on the upper side of the base of the alignment device 4 through the indentations 821, through the channel 48 into the cavity 41, wherein the air previously enclosed therein is guided away through the outlet channel (not illustrated).

FIG. 8 additionally shows an insertion opening 420 for the guide element 30, which opening in the illustrated exemplary embodiment is formed as a slot and ends at the height of the upper side of the base forming the support face 820. A first spacer 490 is arranged above the insertion opening 420, on which spacer the first structural component 10 is rested so that an intermediate space 120 is formed between the first structural component 10 and the guide element 30, which intermediate space continues also toward the front, since a second spacer 402 is formed at the front end and serves as a support face for the first structural component 10. It can be seen from FIG. 8 that the insertion opening 420 is dimensioned so that the lower leaf spring can be pushed through and inserted in a tightly bearing manner. As the adhesive 9 is introduced, adhesive is thus prevented from being able to escape from a region of the insertion opening 420 around the guide element 30. The sealing effect is increased by the pressing of the first structural component 10 against the spacer 401 and therefore against the guide element 30. On account of the second press shoe 72, the support face 820 bears tightly against the guide element 30 so that no adhesive can escape as the cavity 51 is filled.

The front end of the guide element 30 is received completely in the receiving device 4 and is protected and surrounded on all sides: the edging or framing of the upper support face for the first structural component protects the leaf springs at the periphery; the protection on the lower side is provided by the adhesive and the support face on the alignment device 4; merely the upper side is unprotected.

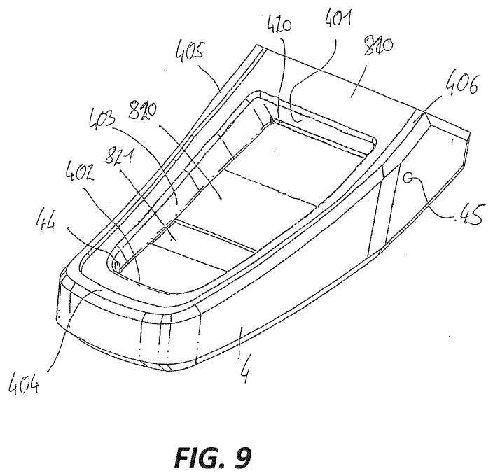

FIG. 9, in a perspective illustration, shows a receiving device 4 in accordance with the embodiment of the previous drawings. Besides the feed connection 44, the outlet channel 45, and the lower support face 820, the indentation 821 is illustrated slightly enlarged. The channel 48, which is fluidically connected to the indentation 821, is not illustrated. The spacers 401, 402 on the rear side and the front side can be seen. The spacers 401, 402 at the same time form, on their upper sides, an upper support face 810 for the first structural component (not illustrated), which is pressed by its lower side against the support face 810. The insertion slot or the insertion opening 420 ends at the height of the lower support face 820. A groove is made in the lateral spacers 403 laterally next to the support face 820, into which groove the leaf-shaped guide element 30 is inserted until it contacts the front termination of the alignment device 4.

The upper support face 810 is edged by side walls 404, 405, 406, which can correspond in terms of their material thickness to that of the upper structural component 10. Due to the side walls 404, 405, 406, a defined assignment of the upper structural component 10 to the alignment device 4 and therefore to the lower guide element 30 is ensured when the front and lateral edges of the structural component 10 bear against the respective side walls 404, 405, 406. If the height of the side walls 404, 405, 406 corresponds to the material thickness of the upper structural component 10, the surfaces can terminate in a flush manner.

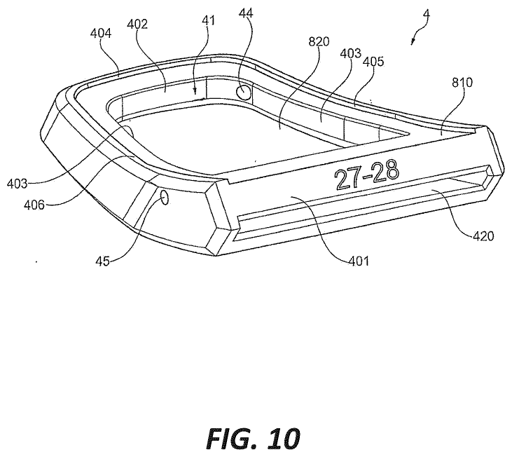

FIG. 10 shows the alignment device 4 in accordance with the previous embodiments in an oblique view from behind, from which the rear spacer 401, the front spacer 402, and the insertion opening 420 are very clearly visible. It can also be seen that the feed connection 44 is lower than the outlet channel 45, wherein both the feed connection 44 and the outlet channel 45 are formed within the spacer 403. A groove, in which the guide element 30 can be inserted, is formed by an undercut in side walls formed below the spacers 403. The elevated side walls 404, 405, 406, which protrude past the upper support face 810, can also be seen, as can the support face 820 on the upper side of the base of the alignment device 4, which support face is flat in the illustrated exemplary embodiment. A receptacle is formed within the alignment device 4 by the side walls 402, 403 and the rear spacer 401, which receptacle can be completely filled with adhesive. By inserting the lower guide element 30 through the insertion opening 420, the insertion opening 420 is closed, so that the receptacle is only open upwardly after the insertion of the guide element 30. If the structural component 10 (not illustrated) is rested on the upper support face 810, the cavity 41 is closed. Once the cavity 41 has been filled with the adhesive, this is connected in an adhesively bonded manner both to the alignment device 4 and to the two leaf springs 10, 30.

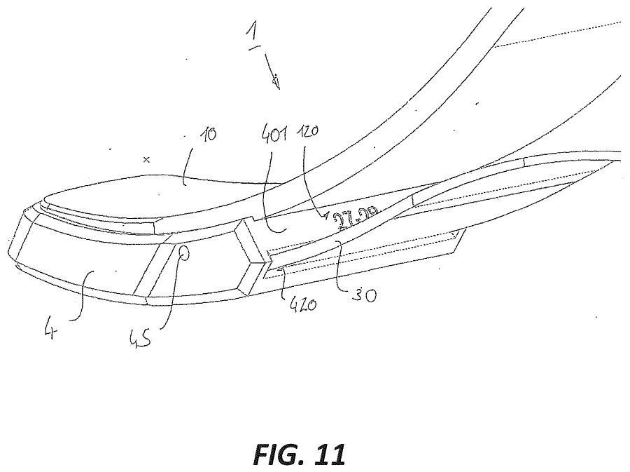

FIG. 11 shows a front part of the orthopedic component in the form of a prosthetic foot 1 obliquely from behind in a finished, assembled state. The guide element 30 is inserted into the insertion opening 420, and the upper structural component 10 is rested and held on the support face 810 (not illustrated), with an intermediate space 12 thus being formed, this being ensured by the spacer 401. The outlet channel is arranged in a side wall, and the components 4, 10, 30 are permanently connected via the adhesive within the receiving device 4.

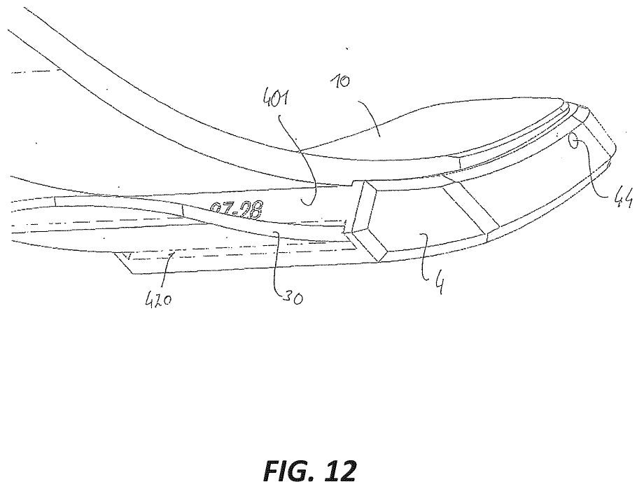

FIG. 12 shows the embodiment according to FIG. 11 from the other side; the feed connection 44 is arranged on a front side wall of the alignment device 4.

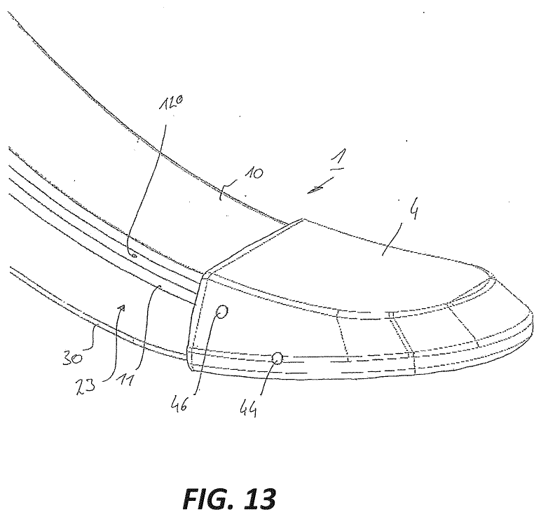

FIG. 13 shows a variant of the invention in which, instead of just two structural components, as is illustrated in FIGS. 6 to 12, three structural components 10, 11, 30 are connected to one another via an alignment device 4. The prosthetic foot 1 is again formed as a prosthetic foot and has a base spring as guide element 30. The forefoot spring is formed as a double leaf spring arrangement connected in parallel, comprising two leaf springs as middle structural component 11 and upper structural component 10. The orientation of the double spring and the base spring corresponds to the orientation as has been described further above; however, different orientations and alignments of the components 10, 11, 30 in relation to one another are, in principle, possible and provided.

An intermediate space 23 is formed between the lower guide element 30 and the second, middle structural component 11, whereas a second intermediate space 120 is formed between the first, upper structural component 10 and the middle structural component 11. The intermediate space is formed by corresponding spacers within the alignment device 4.

The alignment device 4, in contrast to the previous embodiment, is closed upwardly, that is to say the upper structural component 10 is not rested on an upper support face in order to close off a cavity, but rather all structural components and the guide element 30 are inserted into the alignment device 4 from the rear side through insertion openings.

Since the spacing of the respective structural components 10, 11 and of the guide element 30 continues within the alignment device 4, at least two cavities are formed within the alignment device 4 and are separated from one another so that, in the illustrated exemplary embodiment, two feed connections 44, 46 are provided, such that the cavities can be filled separately. It is thus possible to provide for example different adhesives, different adhesive temperatures, or other process features when required by the process.

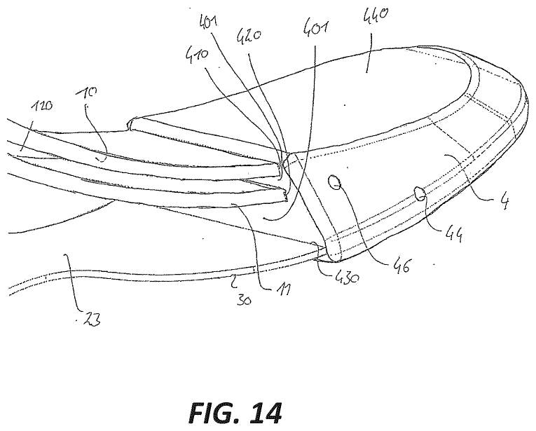

FIG. 14 shows the embodiment according to FIG. 13 in an oblique view from behind. The three insertion openings 410, 420, 430 on the rear end face of the alignment device 4 can be seen, as well as the two feed connections 44, 46 and the rear spacers 401 formed by the rear wall between the leaf springs 10, 11, 30.

The insertion opening 430 for the guide element 30 is arranged, as in the previous embodiment, at the level of the lower support face 820, and the groove, preferably a peripheral groove in the side wall, and an optionally provided structuring of the support face can also be provided. Instead of the upwardly open design, a cover 440 is provided in the illustrated exemplary embodiment according to FIG. 14 so that the upper side of the upper structural component 10 is also covered by the material of the alignment device 4. The front ends of the structural components 10, 20 and of the guide element 30 are thus surrounded completely by the alignment device 4 and are connected to one another and to the alignment device 4 via the adhesive.

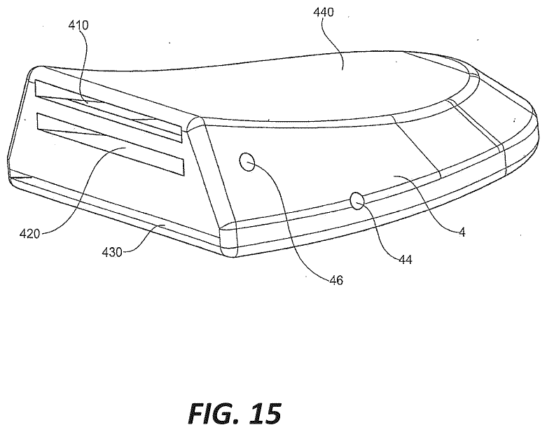

FIG. 15 shows the alignment device 4 in accordance with the second exemplary embodiment in an isolated illustration. The three insertion openings 410, 420, 430 on the rear side can be seen, as well as the two lateral feed connections 44, 46, which identify access to the intermediate spaces or cavities within the alignment device 4 created by the insertion of the leaf springs 10, 11, 30. The upper cover 440 forms the upper termination, and the base of the alignment device 4 forms the lower termination and a sort of sole in an embodiment of the orthopedic component as a prosthetic foot.

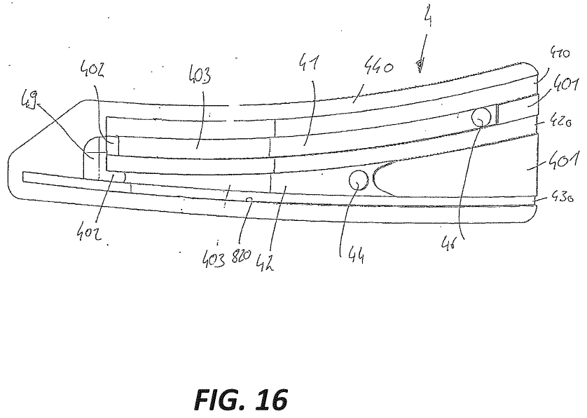

FIG. 16 shows a sectional view of the alignment device 4, from which the insertion openings 410, 420, 430, the rear spacers 401, and the front and lateral spacers 402, 403 can be seen. A channel passing through the front spacers 402 is also formed so that adhesive are admitted, when it are formed by the side through the feed connections 44, 46 into the cavities 41, 42 by the alignment device 4 and the structural components received therein. As an alternative to the embodiment illustrated in FIGS. 13 to 15, it is possible for just the lower opening 44 to be formed as a feed connection, whereas the upper opening is formed as an outlet channel, such that adhesive passes through the feed connection 44, through the cavity 42 and the channel 49, into the cavity 41 and then exits through the outlet channel. The support face 820 can be structured and can also be washed over or wetted by adhesive so that the middle structural component is surrounded both on the lower side and on the upper side by adhesive and is connected thereon on both sides to a different leaf spring. The closed cover 440 can also be seen, as can the closed front tip, and an insertion groove for the lower structural component, which protrudes beyond the channel 49 in the front direction. The feed connections 44, 46 or the feed connection 44 and the outlet channel are formed in the lateral spacers 403.

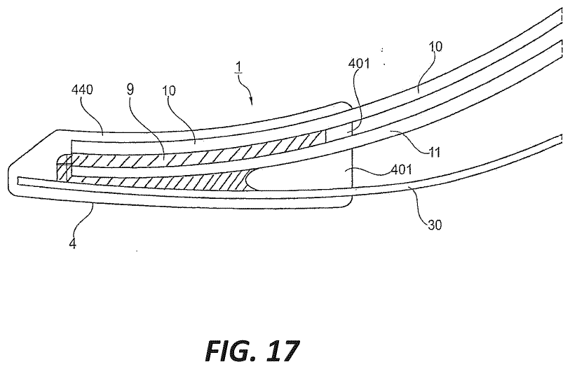

FIG. 17 shows the front end of the prosthetic foot 1 in the assembled state in a schematic sectional illustration. The two structural components 10, 11 and the guide element 30 in the form of leaf springs are inserted through the respective insertion openings into the alignment device 4, and the rear spacers 401, the spacers 403 (not illustrated) and the front spacers 402 are held at a distance from one another in the alignment device 4. The adhesive 9 has been introduced into the cavity 41 through the feed connection 44 (not illustrated), has penetrated through the channel 49 into the upper cavity 42, and has been guided away through the upper outlet channel 45 (not illustrated). No adhesive 9 has escaped rearward during manufacture through the sealing termination of the insertion openings 410, 420, 430 around the leaf springs 10, 11, 30. The adhesive surrounds the second structural component 11 on the upper side, on the front side, and on the lower side.

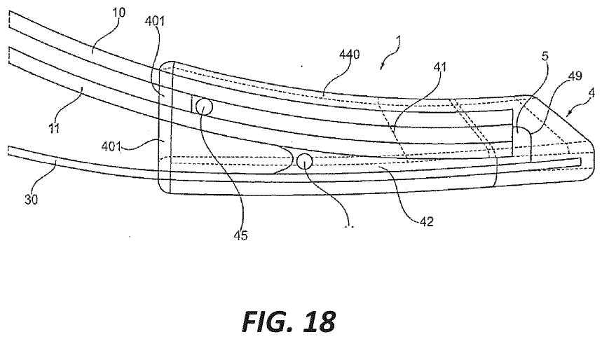

FIG. 18 shows a side view of the assembled prosthetic foot or the prosthetic foot 1, in which case, instead of two feed connections, a lower feed connection 44 and an upper outlet channel 45 are provided in the alignment device 4. The three inserted structural components 10, 11, 30 can also be seen, as can the rear spacers 401, the intermediate spaces or cavities 41, 42, which are sealed to the rear by the inserted structural components 10, 20, 30 and the upper cover 440, by means of which the upper leaf spring or the upper structural component 10 is also covered and protected completely by the alignment device 4.

The adhesive is pushed through the feed connection 44 into the lower cavity 42, through the channel 49 into the upper cavity 41, and out through the outlet channel 45; as soon as adhesive exits from the upwardly placed outlet channel 45, the feed of the adhesive through the feed connection 44 is stopped, the components 10, 11, 30 are held in the desired assignment, and the adhesive is left to cure, such that all components 10, 11, 30, 4 are permanently connected to one another.

further variant of the invention comprises the embodiment in which the alignment device 4 is formed without a base on the lower side. The alignment device 4 is formed here as a frame with support faces for the structural components 10 and guide elements 30 placed above and below. The frame is peripheral with an enclosed opening, which is completed by the leaf springs 10, 30 to form a cavity, into which adhesive 9 is introduced, such that the lower side of the upper structural component 10 and the upper side of the guide element 30 are wetted with adhesive 9 opposite one another and are adhesively bonded to one another at the alignment device 4. Both leaf springs 10, 30 are pressed against the relevant support face and are held pressed until the adhesive 9 has cured, the formed cavity is sealed by pressing against the support faces, excess adhesive 9 exits only through the outlet channel arranged in a spacer, preferably via an outlet device, and therefore the component is not contaminated by adhesive 9. Otherwise, the setup corresponds to that in FIG. 8.

Due to the above-described method, which is also part of the invention, and the orthopedic component according to the invention it is possible to adhesively bond two structural components such as leaf springs, in particular two fiber composite materials, using a liquid adhesive and at the same time to surround these structural components in order to thus provide a protective casing. The alignment device fits on or to the components to be connected and forms a cavity therebetween which forms the receiving space for the liquid adhesive. In order to introduce the adhesive into the cavity or the hollow space and at the same time ventilate the cavity, relatively small openings in the form of feed channels or outlet channels are integrated into the alignment device or the mold and casing. Tube connectors can be inserted into these feed connections and outlet channels and can be connected to a feed tube and a venting tube. In order to ensure that the cavity or the hollow space is reliably sealed, the structural components can be pressed together or can be pressed against the receiving device 4, wherein this can be made possible due to flexible materials. The material of the alignment device 4 is preferably a flexible, resilient material, such that a sealing abutment against the structural components can be ensured by exerting pressure in the direction of the structural components. Once the adhesive has been introduced and cured, the tube connectors are removed from the alignment device or the molding shell and the connection method is complete. The mold now no longer serves as a delimitation for the adhesive; it is used as a shell or casing of the structural components in order to protect the structural components against damage and additionally in order to protect further parts, for example a casing or cosmetic feature against damage by the structural components connected to one another, which can have sharp edges.

Due to the device and the method it is possible to provide a mold for a liquid adhesive for the connection of two structural components. The alignment of the components to be connected is ensured by the alignment device 4, and the component parts to be connected are also protected, the production method is clean, and there is no need for any post-processing of the joint area. The consumption of adhesive is limited, since no excess adhesive can escape, and a defined volume provided by the respective cavities can serve as a basis for the calculation of the fed adhesive quantity. A quantity-controlled feed of adhesive thus ensures that, on the one hand, a minimal quantity of adhesive is used and on the other hand sufficient adhesive is always provided in order to completely fill the cavity.

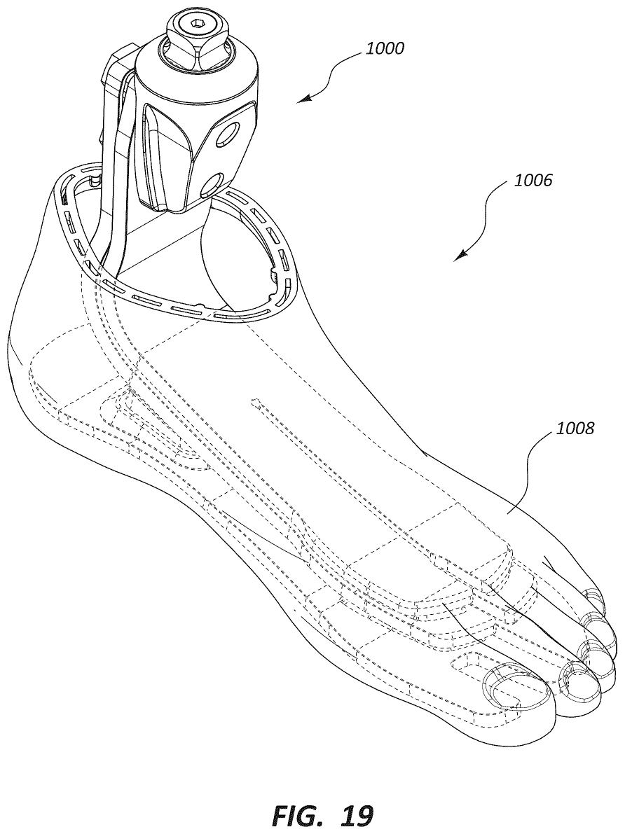

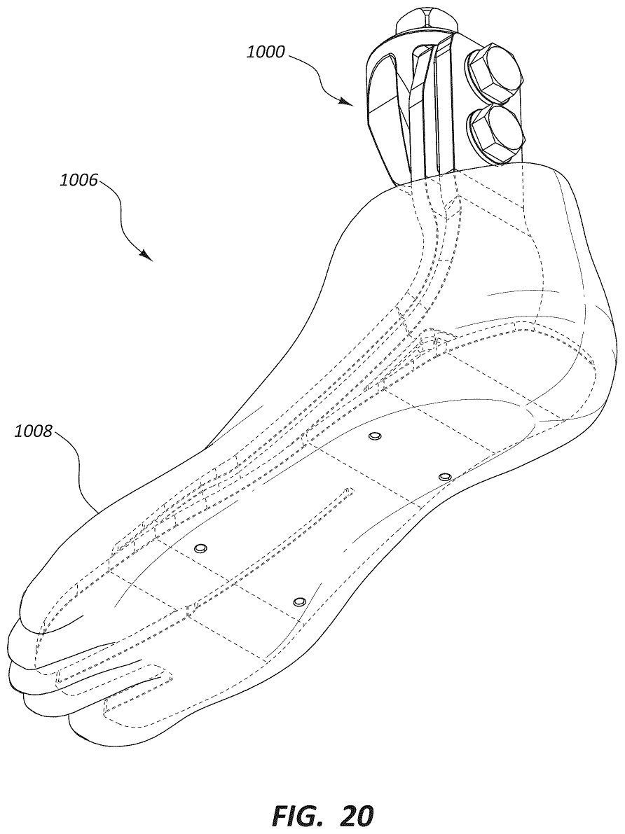

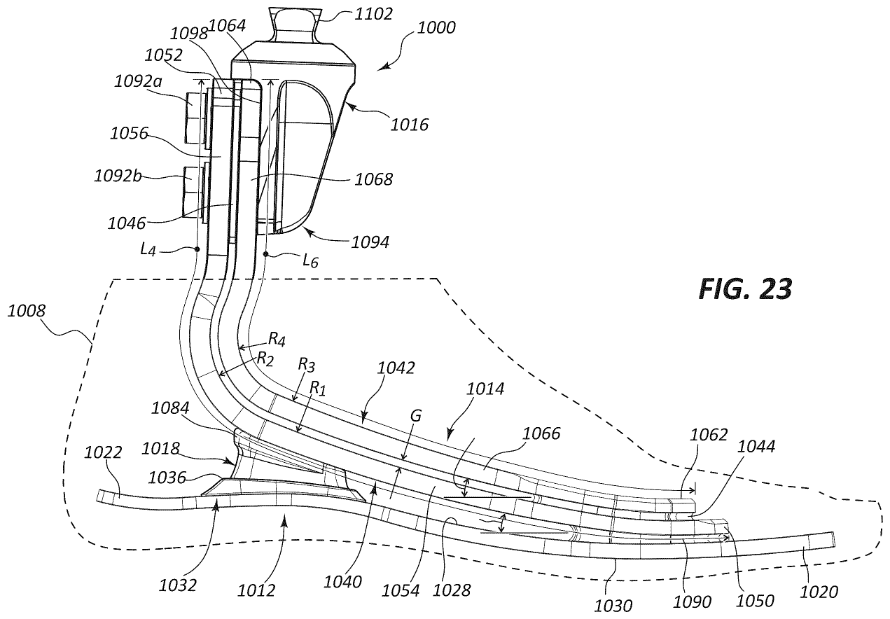

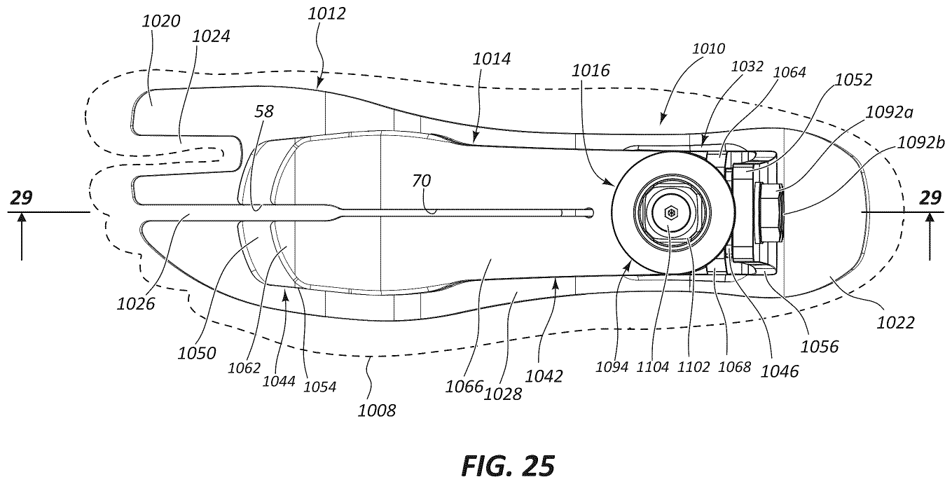

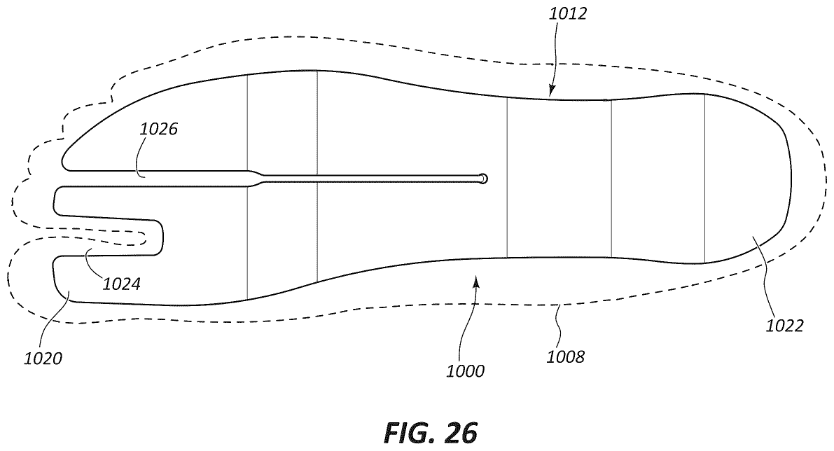

Referring now to FIGS. 19-22, an example prosthetic foot assembly 1006 is shown and described. The prosthetic foot assembly 1006 includes a cosmesis 1008 and a prosthetic foot 1000. The cosmesis 8 is a hollow structure in which portions of the prosthetic foot 1000 are positioned. The cosmesis 1008 provides an aesthetic covering for the prosthetic foot 1000 to give the appearance of an actual foot. FIGS. 23-29 illustrate the prosthetic foot 1000 in further detail. The prosthetic foot 1000 is intended to be used inside a shoe, the heel height being raised to accommodate the heel of a typical shoe.

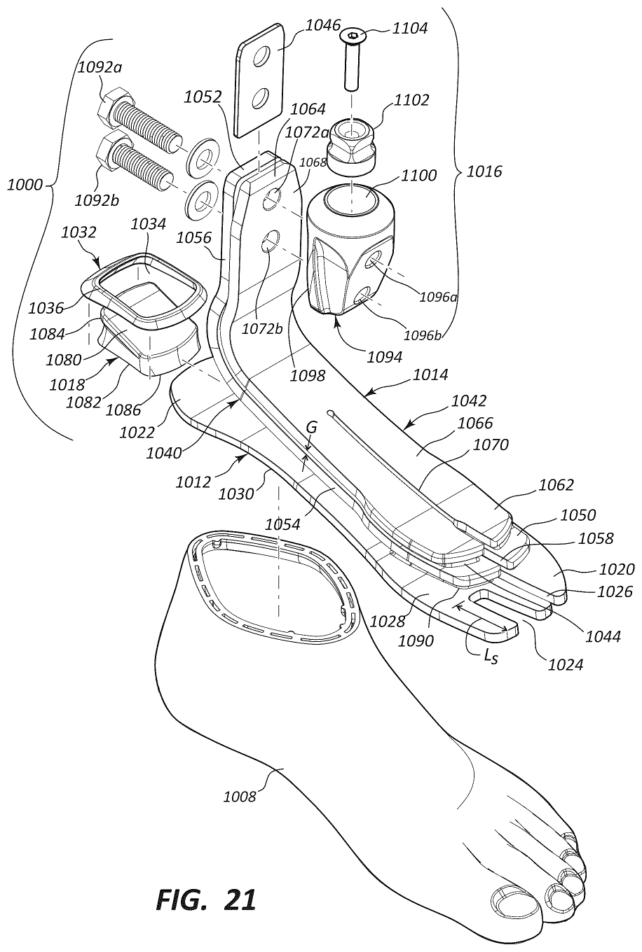

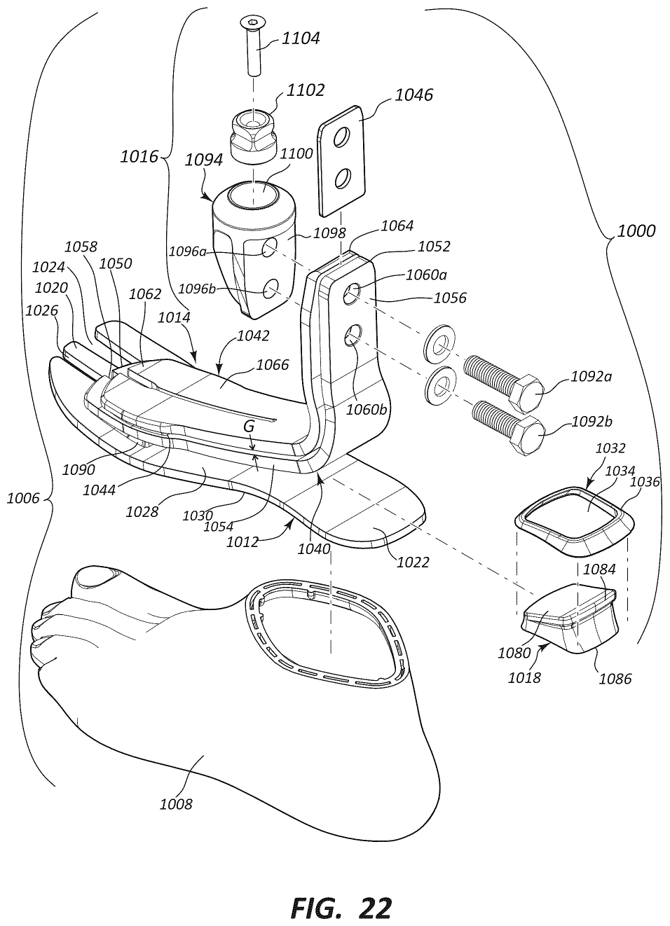

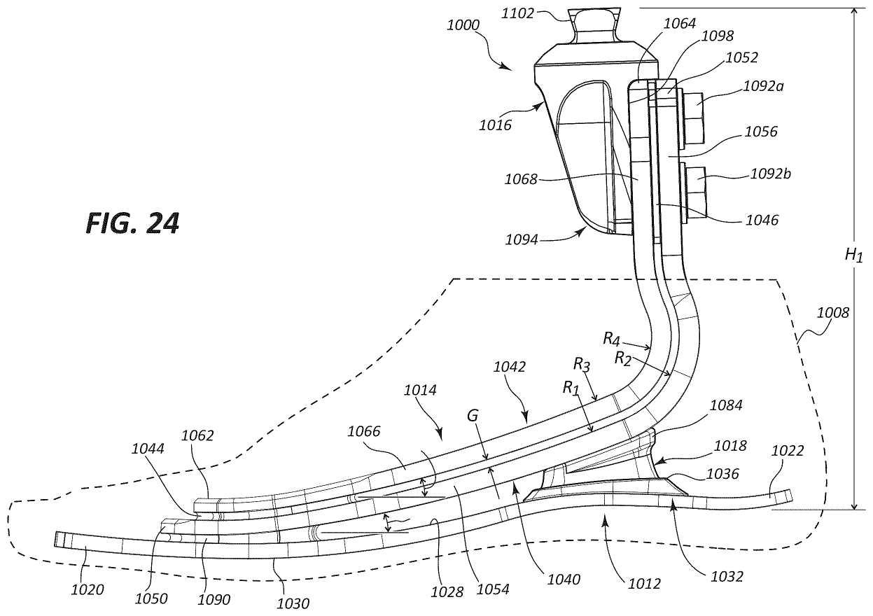

Referring now to FIGS. 21 and 22, the prosthetic foot 1000 is shown including a base spring 1012, a top spring assembly 1014, a connector assembly 1016, and a heel cushion 1018. The top spring assembly 1014 is connected to the base spring 1012 in a toe end area at a toe end connection 1090. The toe end connection 1090 may include a bond connection formed by, for example, an adhesive bond. The toe end connection 1090 may be formed using an elastic, flexible material that provides at least some relative movement between the base spring 1012 and top spring assembly 1014 (e.g., rotational movement about a vertical axis, compression, and translational movement in the anterior/posterior and/or medial/lateral direction). The toe end connection 1090 may provide the sole connection point between the base spring 1012 and top spring assembly 1014. Typically, the heel cushion 1018 is mounted directly to a top surface of the base spring 1012 and arranged to contact a bottom surface of the top spring assembly 1014 as shown in, for example, FIGS. 23 and 24. The heel cushion 1018 may be releasably connected to the base spring 1012. Alternatively, heel cushion 1018 may be releasably connected to the top spring assembly. In at least some examples, the heel cushion 1018 is connected to the base spring 1012 with an interference fit connection using, for example, a retainer 1032 that is mounted to the top surface of the base spring 1012. The heel cushion 1018 may be replaceable with other heel cushions having different properties such as increased or reduced stiffness, compressibility, damping capability, etc. Heel cushions of different sizes and shapes may also be used in place of the heel cushion 1018 shown in the figures. In some examples, the prosthetic foot 1000 may be operable without any heel cushion 1018.

The connector assembly 1016 may be releasably attached to the top spring assembly 1014 at its proximal end. In at least one example, the connector assembly 1016 is releasably connected using one or more fasteners 1092a, 1092b. Connector assemblies with different connector features such as a pyramid connector 1102 may be used. In at least some examples, the pyramid connector is a replaceable component of the connector assembly 1016. In other embodiments, the pyramid connector is integrally formed with remaining portions of the connector assembly and mounted directly to the top spring assembly. Other connector features besides a pyramid connector may be used as part of the connector assembly for securing the prosthetic foot 1000 to another prosthetic member such as a lower leg pylon, a socket, or the like.

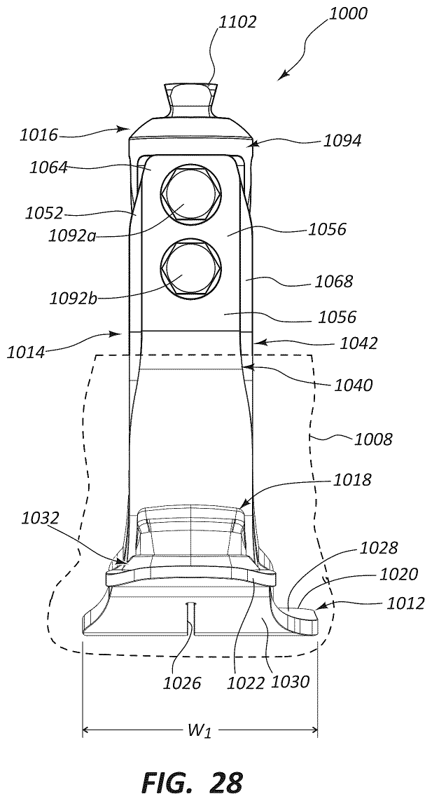

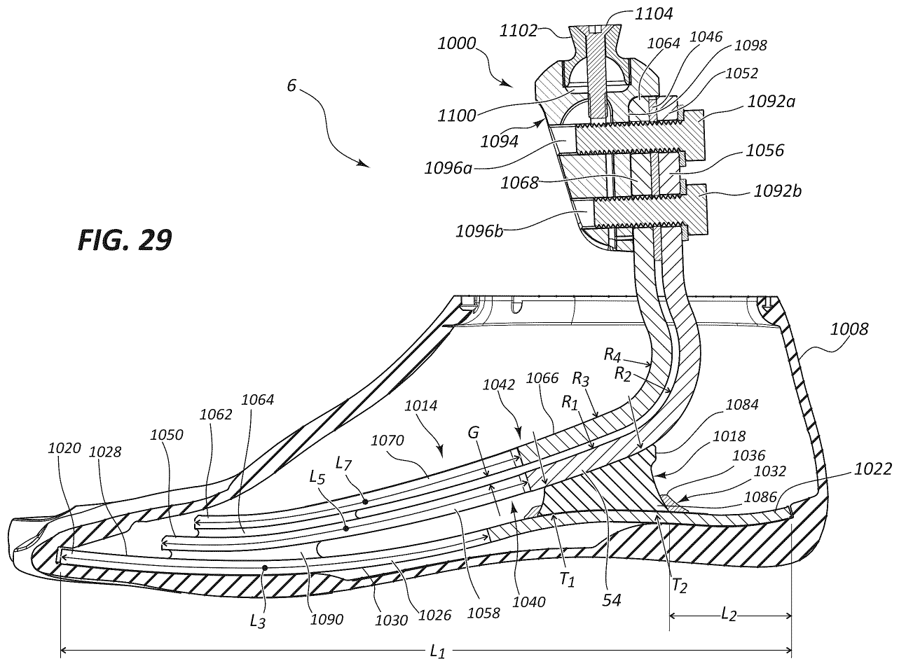

The base spring 1012 is shown including a toe end 1020, a heel end 1022, a sandal slot 1024, and a balance slot 1026. The base spring 1012 may also include a top surface 1028, a bottom surface 1030, and a heel cushion retainer 1032 positioned at a heel end portion of the base spring 1012. The retainer 1032 may include a cavity 1034 and a rim 1036 to help releasably secure the heel cushion 1018 to the base spring 1012. The base spring 1012 may also include a maximum length L₁ (see FIG. 29), a maximum width W₁ (see FIG. 28), a heel lever length L₂ (see FIG. 29) extending posterior of the heel cushion 1018, and a balance slot length L₃ (see FIG. 29). Typically, the maximum length L₁ is in the range of about 7 to about 12 inches, depending on foot size. The maximum width W₁ is typically in the range of about 1 to about 3 inches. The heel lever length L₂ is typically in the range of about 1 to about 3 inches, and more particularly in the range of about 1 to about 2 inches. The L₃ is typically in the range of about 3 to about 8 inches, and more particularly about 5 to about 7 inches. The dimensions of the base spring 1012 may depend largely on the foot size.

The sandal slot 1024 may also have a length Lₛ. The length Lₛ is typically in the range of about 0.5 to about 2 inches. The sandal slot 1024 is formed in the toe end portion of the base spring 1012 and extends posterior from an interior most edge of the base spring 1012. The balance slot 1026 is also formed at the toe end portion beginning at the anterior most edge of the base spring 1012 and extending posteriorly. In at least some embodiments, the balance slot 1026 is aligned with a longitudinal center line of the base spring 1012. The balance slot 1026 may provide enhanced medial/lateral compliance for the prosthetic foot 1000, particularly when walking on uneven surfaces.

As shown in at least FIGS. 23 and 24, the base spring 1012 has a contoured shape along its length. The side profile of the base spring 1012 undulates between concave and convex shapes. In some examples, the distal surface of the base spring 1012 is preferably convex in an anterior section, transitions to concave in an arch or mid-section, and may transition back to convex at the posterior end. These contours and the location of the contours, particularly relative to the toe end connection 1090 and the heel cushion 1018, may provide improved rollover smoothness, enhanced energy feedback to the user, stability, and comfort during use of the prosthetic foot. Providing the lever portion extending posterior of the heel cushion 1018 may also provide improved smoothness in the rollover and energy feedback during use.

The top spring assembly 1014 is shown including first and second spring members 1040, 1042, a first spacer 1044 at the toe end portion of the prosthetic foot, a second spacer 1046 positioned at a proximal end of the top spring assembly 1014 and a gap G provided between the first and second spring members 1040, 1042 along their entire length. The first and second spring members 1040, 1042 may be referred to as leaf springs. The first and second spring members 1040, 1042 may extend generally in parallel with each other along their entire lengths. The first spacer 1044 may be provided as a bond connection between the first and second spring members 1040, 1042. In at least some examples, the first spacer 1044 comprises the same bond material as used for the toe end connection 1090 between the top spring assembly 1014 and the base spring 1012. In at least some embodiments, the first spacer 1044 is positioned generally in alignment with the toe end connection 1090 so as to be positioned vertically above the toe end connection 1090, or at least partially overlapping the toe end connection 1090 in a length dimension of the base spring 1012. The first spacer 1044 may provide a permanent connection between the first and second spring members 1040, 1042. The material of first spacer 1044 may provide at least some relative movement between the first and second spring members 1040, 1042 (i.e., rotational movement about a vertical axis, translational movement in an anterior, posterior or medial/lateral direction, compression, etc.). The material of first spacer 1044 may be elastic so as to return to its original shape upon removal of a force that is used to compress or deform the first spacer 1044.

In other examples, the first spacer may comprise a wear resistant, low friction material that is attached to one of the first and second springs. The first spacer is not attached or connected to the other of the first and second springs. This arrangement supports compression forces between the distal ends of the first and second springs and allows the springs to separate during plantarflexion and also slide against each other at the distal ends of the springs. Such an embodiment may also alter performance of the foot during rollover in comparison to having the first spacer as a bond connection. Tensile and shear forces are not transferred through the spacer, hence the deflection and stress conditions in the upper spring assembly are modified. The first spring is in an unloaded condition during plantarflexion at heel strike and, as the foot rolls over and the user's weight is transferred to the toe, shear displacement between the distal ends of the first and second springs results in increased defection the foot in the toe region, thereby softening the foot during both the heel strike and terminal stance portions of the gait cycle

The second spacer 1046 may comprise a rigid material that is non-compressible and/or non-elastic. The second spacer 1046 may be positioned at a proximal most end of the top spring assembly 1014. The second spacer 1046 may be aligned with the connector assembly 1016, or at least portions thereof. In the illustrated embodiment, the second spacer 1046 includes apertures through which the fasteners 1092a, 1092b extend for connection of the connector assembly 1016 to the top spring assembly 1014.

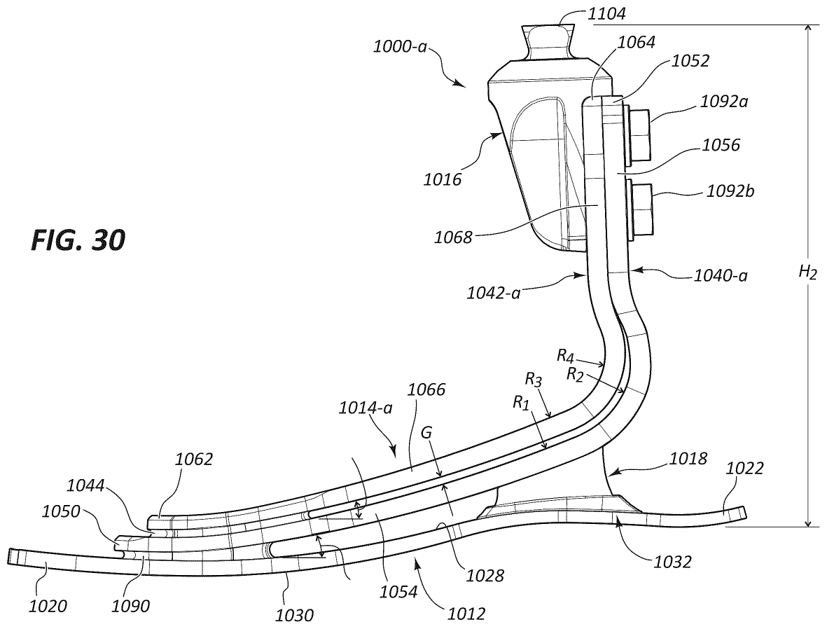

The first and second spacers 1044, 1046 may define the size of the gap G when the prosthetic foot 1000 is in a rest state. Typically, the gap G is provided along an entire length of the first and second spring members 1040, 1042 when the prosthetic foot 1000 is in a rest state (i.e., prior to application of a force during use of the prosthetic foot 1000). Alternatively, the two upper springs 1040, 1042 may abut (e.g., directly contact each other) at the connector location as shown in FIG. 30. The gap G may vary in size during operation of the prosthetic foot 1000. For example, the gap G may reduce in size at the first spacer 1044 if the material of the first spacer 1044 is compressible during use. In another example, the gap G may reduce or change size at locations between the first and second spacers 1044, 1046 during use. For example, applying a force from a user during a gait cycle may change the size of gap G at various phases of the gait cycle (e.g., at heel strike, stance phase, and toe off), as the forces are applied and released during use by a wearer, those forces are absorbed and/or fed back through the base spring 1012 and heel cushion 1018. In at least some embodiments, the first spring member 1040 may come into contact with the second spring member 1042 during use of the prosthetic foot (i.e., the gap reduces to zero).

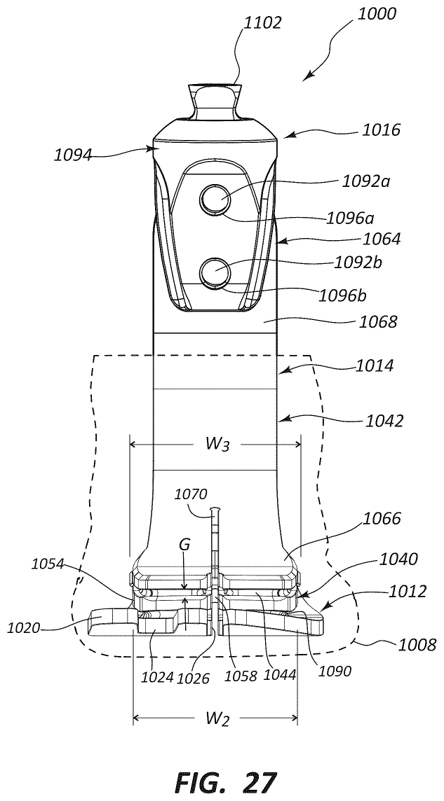

The first spring member 1040 is shown having an anterior end 1050, a proximal end 1052, a horizontal portion 1054, a vertical portion 1056, a slot 1058, and fastener apertures 1068a, 1060b. The first spring member 1040 has an overall length L₄ as shown in FIG. 23, a maximum width W₂ as shown in FIG. 27, and a length L₅ for slot 1058 as shown in FIG. 29. The horizontal portion 1054 may have a radius of curvature R₁ as shown in FIGS. 23 and 24. The first spring member 1040 also has a bend radius R₂ at the intersection between the horizontal and vertical portions 1054, 1056. The radius of curvature R₁ may be variable along the length from the anterior end 1050 in a posterior direction. The radius R₁ is typically in the range of about 10 to about 30 inches, and more particularly about 18 to about 22 inches. The bend radius R₂ is typically in the range of about 0.75 to about 3 inches, and more particularly about 1 to about 1.5 inches. Although the horizontal portion 1054 extends generally in a horizontal direction, it may be arranged at an angle α relative to a horizontal plane as shown in at least FIG. 23. The angle α may be measured along that part of the horizontal portion 1054 that is posterior of the first spacer 1044. The angle α is typically in range of about 2° to about 30°, and more particularly about 5° to about 15°.

The second spring member 1042 may include an anterior end 1062, a proximal end 1064, a horizontal portion 1066, a vertical portion 1068, a slot 1070, and fastener apertures 1072a, 1072b. The second spring member 1042 may have an overall length L₆ as shown in FIG. 23, a maximum width W₃ as shown in FIG. 28, and a length L₇ for slot 1070 as shown in FIG. 29. The slot 1070 may be formed at the anterior end 1062 and extend posteriorly. The slot 1072 may be aligned with the slot 1058 of the first spring member 1040 and the balance slot 1026 formed in base spring 1012. In at least some examples, the slots 1026, 1058, 1070 may extend in a posterior direction to a common location. The slots 1026, 1058, 1070 may terminate at different locations in the anterior direction as shown in at least FIG. 21. The slots 1058, 1070 may be aligned with a center line of the base spring 1012 and top spring assembly 1014 so as to provide balanced medial/lateral pronation and compliance during use of the prosthetic foot.

The first spring member 1040 may include a radius of curvature R₄ for the horizontal portion 1066 along its length. The radius R₃ may be variable along its length from the anterior end 1062 toward the vertical portion 1068. The second spring member 1042 may also include a bend radius R₄. Typically, the radiuses R₃, R₄ are similar to the radiuses R₁, R₂. Radius R₃ may be in the range of about 10 to about 30 inches, and more particularly about 18 to about 22 inches. R₄ may be in the range of about 0.75 to about 3 inches, and more particularly about 0.5 to about 1 inches. Because the second spring member 1042 is positioned on an upper side and nested within the concave curvature of the first spring member 1040, the radius R₄ is typically smaller than the radius R₂.

The horizontal portion 1066 may be arranged at an angle θ relative to a horizontal plane as shown in FIG. 23. The angle θ may be in the range of about 2° to about 30°, and more particularly about 5° to about 20°. The angle θ typically is the same or similar to the angle α for horizontal portion 1054.

The top spring assembly 1014 is mounted to the base spring 1012 as shown in at least FIGS. 23-29. The heel cushion 1018 is arranged to contact a bottom or downward facing side or surface of the top spring assembly 1014 (e.g., a bottom surface of first spring member 1040 as shown in FIG. 23). Although the heel cushion 1018 is shown connected to the base spring 1012 and not the top spring assembly 1014, other embodiments may provide the heel cushion 1018 connected to both the base spring 1012 and top spring assembly 1014, or connected only to the top spring assembly 1014 (e.g., the retainer 1032 is mounted to the bottom surface of first spring member 1040 for releasable attachment of the heel cushion 1018).

The heel cushion 1018 may be releasably mounted to the base spring 1012 (or top spring assembly 1014). Alternatively, the heel cushion 1018 may be permanently connected to the base spring 1012. The replaceability of heel cushion 1018 may provide customization of the amount of cushioning, energy dampening, and the like provided by heel cushion 1018. Heel cushion 1018 may be connected with an interference fit connection. Other embodiments may provide for the heel cushion 1018 to be secured with a positive connection such as, for example, a fastener, clip, bracket or the like.

The heel cushion 1018 may include a top surface 1080 (see FIGS. 21 and 22), a tapered shape having a variable thickness along its length, a bottom surface 1082, and top and bottom perimeter rims 1084, 1086. The tapered shape may provide for a smaller thickness T₁ at an anterior end as compared to a greater thickness T₂ at a posterior end of the heel cushion 1018, as shown in FIG. 29. The tapered shape of the heel cushion 1018 may match the angle α and/or curvature R₁ of the first spring member 1040. As such, the top surface 1080 may have a contoured shape rather than a planar shape. Similarly, the bottom surface 1082 may have a shape that matches the contour or curvature of the top surface of the base spring 1012, as shown in at least FIG. 29.

The heel cushion 1018 may comprise a shock absorbing, dampening material such as, for example, silicone or urethane elastomers including, for example, silicone or urethane foams. In some embodiments, the heel cushion 1018 may include a plurality of different materials, layers of materials, or separate components that are secured together as an assembly to provide the desired cushioning properties. In one example, the heel cushion 1018 includes a foam material encapsulated within a protective polymer shell. In another example, the heel cushion 1018 includes a gel material or capsule that is encapsulated within a foam material.

The connector assembly 1016 includes a base 1094, fastener bores 1096a, 1096b, a spring assembly seat 1098, a bore 1100, a pyramid connector 1102, and a fastener 1104. The fastener bores 1096a, 1096b are receptive of the fasteners 1092a, 1092b. In at least some examples, the fastener bores 1096a, 1096b include threads that threadably engage with threads of the fasteners 1092a, 1092b. In other examples, the fastener bores 1096a, 1096b are pass-through bores and the fasteners 1092a, 1092b extend through the bores and threadably engage with nuts positioned on an opposite side of the base 1094. The spring assembly seat 1098 may be shaped and sized to interface with one or both of the first and second spring members 1040, 1042. The spring assembly seat 1098 may include a lip or edge that engages a proximal most surface of, for example, the second spring member 1042 at the proximal end 1064. This lip or edge may provide a more secure connection and interface between the connector assembly 1016 and the top spring assembly 1014.

The bore 1100 may be sized to receive a portion of the pyramid connector 1102. The bore 1100 may include a first portion sized to receive the pyramid connector 1102 and a second portion (e.g., a threaded bore) that threadably engages with threads of the fastener 1104. The bore 1100 may be sized to receive pyramid connectors 1102 of different sizes and shapes, or different types of connectors that may be used to secure the prosthetic foot 1000 to a separate lower leg prosthetic member.

The base spring 1012 and first and second spring members 1040, 1042 may comprise a fiber reinforced composite material such as, for example, carbon fiber reinforced composite. The first spacer 1044 may include an adhesive bond comprising a flexible adhesive such as, for example, a urethane adhesive having a Shore A hardness in the range of about 70 to about 95. During manufacture of the top spring assembly 1014, the first and second spring members 1040, 1042 may be bonded together using a removable gasket between the springs to create a sealed space for the adhesive, and the adhesive is then injected into the space.

The second spring member 1042 may be shorter in length L₅ than the length L₄ for the first spring member 1040. This difference in length may allow for a somewhat gradual change in stiffness in the top spring assembly 1014. Although two spring members 1040, 1042 are shown as part of the top spring assembly 1014, other embodiments may utilize more than two leaf spring elements, and the leaf spring elements may have the same or different lengths.

The second spacer 1046 may comprise a lightweight material such as, for example, aluminum, nylon or fiberglass sheet material (e.g., fiberglass G-10). The top spring assembly 1014 may provide a connection between the first and second spring members 1040, 1042 at opposite ends with a gap G provided there between, thereby providing a number of unexpected structural advantages. These advantages in connection with the type of spacers 1044, 1046, the toe end connection 1090, the heel cushion 1018, and/or other features may provide a number of performance advantages as compared to known prosthetic feet. For example, a dual, narrow cantilever beam, one located above the other and with a space in between the upper and lower beams, and with frictionless spacer at the free end to transmit an applied vertical force from the upper beam to the lower beam at the free end, may result in about 15-25% reduction in bending stress and about 30-45% reduction in shear stress as compared to an equivalent stiffness single cantilever beam. If the first spacer is comprised of a low friction material connected to one of the first and second springs, the boundary conditions described are highly accurate. If the first spacer is a bond connection (e.g., created with a flexible material), the boundary conditions are approximately midway between a frictionless spacer between the distal ends of the first and second spring and a rigid connection at the distal ends of the first and second springs. Because stresses are reduced by using the dual upper spring design, a prosthetic foot utilizing this dual spring design exhibits at least one of improved durability and improved flexibility as compared to single spring designs and dual spring designs which are rigidly connected at the distal end. Furthermore, utilizing a low friction spacer material may provide more flexibility than utilizing a flexible bond connection, thus potentially providing opportunities to achieve different and desirable performance characteristics and multiple design options to achieve the designer's goal. Many of the advantages of the dual cantilever beam designs disclosed herein may be maximized when the two beams (e.g., first and second spring members 1040, 1042) have substantially equal bending stiffness. If the beams are constructed of unidirectional fiber reinforced composite lamina, the maximum strength/stiffness ratio may be best achieved when both beams have substantially the same lamina orientation and thickness. As the difference between the bending stiffness of the upper and lower beams increases, the advantages of a dual cantilever spring design typically diminish.

The heel cushion 1018 may comprise a silicone or urethane elastomer (e.g., an elastomer with the Shore hardness range of about 50A to about 90A). The heel cushion 1018 may be retained with retainer 1032 in a way that extends around an entire perimeter of the heel cushion 1018. Other embodiments may provide for a retainer that extends around only a portion of perimeter of the heel cushion 1018. The retainer 1032 may be bonded to the top surface 1028 of the base spring 1012 using, for example, an adhesive. In some embodiments, both the adhesive and the retainer 1032 are somewhat flexible to avoid detachment of the retainer 1032 from the base spring 1012 when the base spring 1012 flexes during use. The retainer 1032 and adhesive may comprise a plastic material having a Shore hardness in the range of, for example, about 90A to about 50D. Alternatively, the retainer 1032 may be cast into the structure of base spring 1012 along the top surface 1028 thereof, which may eliminate the need for use of an adhesive or other bonding agent.

The retainer 1032 may help keep the heel cushion 1018 in place by utilizing geometric interlocking features. These interlocking features may include angled (e.g., wedge-shaped) features in the retainer and along an exterior of the heel cushion 1018, wherein corresponding surfaces interface to provide a connection The heel cushion 1018 may be deformed or compressed in order to fit into the interior of the retainer 1032, and then expanded automatically to its original shape thereby creating an interference fit connection between the features of the retainer 1032 and the heel cushion 1018. Alternatively, the retainer 1032 and the heel cushion utilize a rib that fits into a recess, wherein the rib and recess may be formed on either the retainer 1032 or heel cushion 1018.

Generally, the base spring 1012 extends from the toe region to a heel region of the prosthetic foot. The base spring 1012 may extend from an interior most point of the prosthetic foot to a posterior most point of the prosthetic foot. The top spring assembly 1014 may be connected to the base spring 1012A at a location spaced anterior of an anterior most edge of the base spring 1012. In at least one example, the top spring assembly 1014 is positioned posterior of the sandal slot 1024 formed at the distal end of the base spring 1012. The base spring 1012 may extend in an anterior direction at least as far as an anterior most point along a length of the top spring assembly 1014.

As discussed above, the slot or split 1026 formed in the base spring 1012 from the anterior edge in a posterior direction may be aligned with the slots or slits 1058, 1070 formed in the top spring assembly 1014 from the anterior end of the top spring assembly 1014 extending in a posterior direction. These slots or splits may provide for the entire prosthetic foot 1000 to be divided into medial and lateral sides at least in the toe and midfoot regions of the prosthetic foot.

The top spring assembly 1014 includes first and second spring members 1040, 1042 that extend to different anterior positions along the length of the prosthetic foot. At least FIG. 23 illustrates the first spring member 1040 extending further in an anterior direction than the second spring member 1042. The first spacer 1044 is positioned at the anterior most edge of the second spring member and spaced posterior of the anterior most edge of the first spring member 1040.

The top spring assembly 1014 extends generally parallel with the base spring 1012 in the toe and midfoot regions of the base spring 1012. The top spring assembly 1014 may extend in a generally vertical direction relative to the base spring 1012 in the heel end portion of the prosthetic foot. As described above, other embodiments may provide for the top spring assembly 1014 to continue extending in a generally horizontal or slightly angled direction relative to the base spring 1012 and/or a horizontal plane through the heel end portion.

Referring now to FIG. 30, another example prosthetic foot 1000-a is shown. The prosthetic foot 1000-a includes the same or similar components as the prosthetic foot 1000 described above with reference to FIGS. 19-29 with exception of the top spring assembly 1014-a. The top spring assembly 1014-a does not include second spacer 1046 at its proximal end. The first and second spring members 1040-a, 1042-a of the top spring assembly 1014-a are arranged in direct contact with each other at the proximal end. The elimination of spacer 1046 may result in several changes in the top spring assembly 1014-a. For example, the radius of curvature R₂ of the first spring member 1040-a may be smaller than the radius of curvature R₂ of first spring member 1040 shown in FIGS. 19-29. The radius of curvature R₄ of the second spring member 1042-a may be greater than the radius of curvature R₄ of second spring member 1042 shown in FIGS. 19-29. Removal of the spacer 1046 may also alter the size of radius of curvatures R₁ and R₃ as compared to those values for first and second spring members 1040, 1042 shown in FIGS. 19-29 (e.g., reduce R₁ and increase R₃).

Additionally, the gap G provided between the first and second spring members 1040-a, 1042-a may vary along the length of the first and second spring members 1040-a, 1042-a from the first spacer 1044 toward the proximal end of top spring assembly 1014-a where the first and second spring members 1040-a, 1042-a are in direct contact with each other. The first and second spring members 1040-a, 1042-a may extend generally in parallel with each other along the portions 1054, 1066 to provide a substantially constant gap G in that portion of the top spring assembly 1014-a, have a greater or smaller gap G in the area of radius of curvatures R₂, R₄, and then have a tapered gap G to the point of contact between the end portions 1056, 1068.

The gap G may extend along only a portion of the length of the first and/or second spring members 1040-a, 1042-a. For example, the gap G may be provided along a percentage of the length L₄ of the first spring member 1040-a, such as in the range of about 60% to about 90% of the length L₄. During use of the prosthetic foot 1000-a, portions of the first and second spring members 1040-a, 1042-a may move toward and/or away from each other to alter the size of gap G at various locations along the length of the top spring assembly 1014-a. In at least some embodiments, portions of the first and second spring members 1040-a, 1042-a may contact each other at locations spaced distal of where the first and second spring members 1040-a, 1042-a are shown contacting each other in FIG. 30.

The first spacer 1044 may be provided as a bond connection between the first and second spring members 1040-a, 1042-a. In at least some examples, the first spacer 1044 may comprise a relatively incompressible material such as those described above related to spacer 1046. The first spacer 1044 may be provided as a separate piece that is secured in place between the first and second spring members 1040-a, 1042-a using an adhesive or other bonding agent. The material of first spacer 1044 may provide at least some relative movement between the first and second spring members 1040-a, 1042-a (i.e., rotational movement about a vertical axis, shear displacement or translational movement in an anterior, posterior or medial/lateral direction, compression, etc.). The material of first spacer 1044 may be elastic so as to return to its original shape upon removal of a force that is used to compress or deform the first spacer 1044.

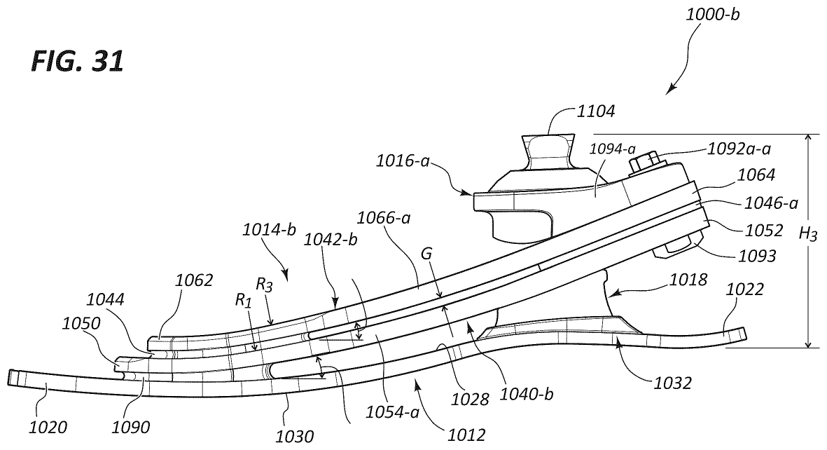

Referring now to FIG. 31, another example prosthetic foot 1000-b is shown. The prosthetic foot 1000-b includes many of the same or similar components as the prosthetic foot 1000 and the prosthetic foot 1000-a described above with reference to FIGS. 19-30 with exception of the top spring assembly 1014-b. The top spring assembly 1014-b does not include vertical portions 1056, 1068 as do the top spring assemblies 1014 and 1014-a. The first and second spring members 1040-b, 1042-b of the top spring assembly 1014-b are arranged substantially horizontally along their length from their anterior ends 1050, 1062 to their proximal ends 1052, 1064. The elimination of vertical portions 1056, 1068 from the first and second spring members 1040-b, 1042-b may result in several changes in the top spring assembly 1014-b as compared to the embodiments shown in FIGS. 19-30. For example, the horizontal portion 1054-a, 1066-a of the first and second spring members 1040-b, 1042-b may be longer than the horizontal portions 1054, 1066a of the first and second spring members 1040, 1042 shown in FIGS. 19-30. The horizontal portions 1054-a, 1066-a may extend from the anterior ends 1050, 1062 to the proximal ends 1052, 1064.

Additionally, the gap G provided between the first and second spring members 1040-b, 1042-b may have a reduced length from the first spacer 1044 to the second spacer 1046. The gap G may be substantially constant when the prosthetic foot 1000-b is in a rest or unloaded state. During use of the prosthetic foot 1000-b, portions of the first and second spring members 1040-b, 1042-b may move toward and/or away from each other to alter the size of gap G at various locations along the length of the top spring assembly 1014-b. In at least some embodiments, portions of the first and second spring members 1040-b, 1042-b may contact each other.

The first spacer 1044 may be provided as a bond connection between the first and second spring members 1040-b, 1042-b. In at least some examples, the first spacer 1044 may comprise a relatively incompressible material such as those described above related to spacer 1046. The first spacer 1044 may be provided as a separate piece that is secured in place between the first and second spring members 1040-b, 1042-b using an adhesive or other bonding agent. In other examples, material of first spacer 1044 may provide at least some relative movement between the first and second spring members 1040-b, 1042-b (i.e., rotational movement about a vertical axis, shear displacement or translational movement in an anterior, posterior or medial/lateral direction, compression, etc.). The material of first spacer 1044 may be deformable and/or elastic so as to return to its original shape upon removal of a force that is used to compress or deform the first spacer 1044.

The second spacer 1046-a may have many of the same or similar features and properties of second spacer 1046 described above. The second spacer 1046-a may have a different arrangement of holes to receive one or more fasteners 1092a-a. In some example, the prosthetic foot 1000-b may include a plurality of fasteners 1092a-a to secure connector assembly 1016-a to the top spring assembly 1014. A base 1094-a of the connector assembly 1016-a may include one or more fastener holes to receive the one or more fasteners 1092a-a. The base 1094-a may be sized and arranged to orient the pyramid connector 1102 in a vertical orientation when the prosthetic foot is unloaded, as shown in FIG. 31.

The fasteners 1092a-a may be arranged side-by-side in a medial/lateral direction. In other arrangements, the fasteners 1092a-a may be arranged in alignment with a length dimension of the prosthetic foot 1000-b. Although only one fastener 1092a-a is shown in FIG. 31, a plurality of fasteners 1092a-a may be used. The fasteners 1092a-a may provide a positive connection between the first and second spring members 1040b, 1042-b, a positive connection between the top spring assembly 1014-b and the connector assembly 1016-a, and/or a positive connection between one or both of the first and second spring members 1040-b, 1042-b and the spacer 1046-a. In some examples, the fasteners 1092a-a are connected directly to one or both of the first and second spring members 1040-b, 1042-b (e.g., to a threaded seat formed in one or both of the first and second spring members 1040-b, 1042-b), or may be connected to a nut 1093 positioned on an opposite side of the top spring assembly 1014-b, as shown in FIG. 31.

As is possible with the prosthetic feet 1000, 1000-a, the heel cushion 1018 may be positioned between the base spring 1012 and the top spring assembly 1014-b. In at least some embodiments, the heel cushion 1018 is mounted to the base spring 1012, and may be releasably mounted to the base spring 1012. The heel cushion 1018 is arranged to contact a bottom surface of the top spring assembly 1014-b, such as a long a bottom surface of the first spring member 1040-b.

An overall height H₃ of the prosthetic foot 1000-b may be substantially smaller than the heights H₁ and H₂ of the prosthetic feet 1000, 1000-a, respectively. The smaller height H₃ may provide a relatively low profile for the prosthetic foot 1000-b and the prosthetic foot 1000-b may be referred to as a low profile prosthetic foot. The prosthetic feet 1000, 1000-a may be referred to as high profile or medium profile prosthetic feet.

The prosthetic foot 1000-b may provide many of the same or similar functions and benefits related to performance and user comfort as described herein related to prosthetic feet 1000, 1000-a. For example, the prosthetic foot 1000-b may provide energy feedback, stability, force dampening and the like associated with the use of spaced apart spring members in the top spring assembly 1014-b, the use of a heel cushion 1018 arranged in the specific location and having the size and shape shown in FIG. 31, the shape and size of the top spring assembly 1014-b and base spring 1012, and the size, shape and orientation of the connector assembly 1016-a. Furthermore, the base spring 1012 and top spring assembly 1014-b may include slots (e.g., slot 1026 for base spring 1012 and slots 1058, 1070 for first and second spring members 1040-b, 1042-b) that provide medial/lateral pronation and ambulation for the prosthetic foot 1000-b, which may provide improved stability for the user, particularly on uneven ground surfaces.

The prosthetic feet 1000, 1000-a, 1000-b may be referred as dual or multiple toe spring prosthetic feet. The prosthetic feet 1000, 1000-a, 1000-b may be referred to as a single toe spring prosthetic feet. The heel assemblies, attachment assemblies, connection assemblies, and other features disclosed with reference to any single embodiment disclosed herein may be interchangeable with features of other prosthetic foot embodiments disclosed herein.

In alternative embodiments, the connection between the base spring and the top spring assembly and between the first and second spring members in the anterior region of the foot and may be provided with bolts or other fasteners. A rigid spacer may be provided between the spring members and/or between the top spring assembly and the base spring. The use of bolts or other fasteners in combination with an altered geometry of the first and second spring members may eliminate gaps that may otherwise exist at connection points at the anterior end of the prosthetic foot. In another embodiment, a connection between the first and second spring members may be made by wrapping carbon fiber or glass fiber around the first and second spring members at the connection point between the first and second spring members, and securing the spring members and the fiber by impregnating the fiber with epoxy or similar thermosetting resin. A similar connection may be made between the top spring assembly and the base spring.

In another example, the connection at the proximal end of the top spring assembly may be created by altering a geometry of the first and second spring members such that no gap exists at the connection points between the first and second springs (e.g., as shown in FIG. 30). In this arrangement, a gap may still be provided between the first and second spring members at other locations along their lengths (e.g., as shown in FIG. 30). In some embodiments, one or more of the first and second spring members may be inserted into a slot formed in the prosthetic connector (e.g., base 1094 of connector assembly 1016), and the first and second spring members are secured together and to the prosthetic connector with an adhesive or a fastener.

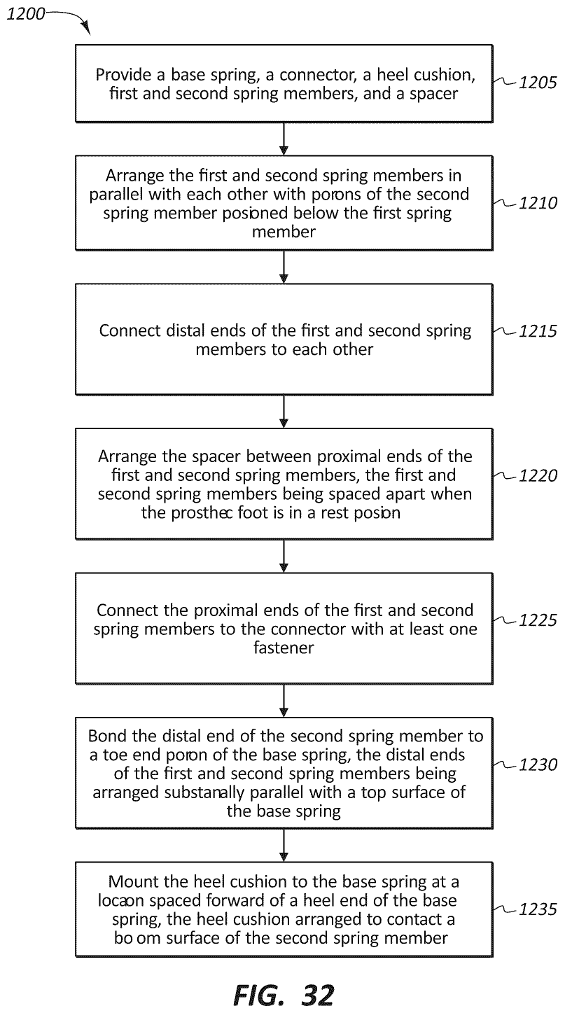

Referring now to FIG. 32, an example method 1200 of manufacturing and/or assembling a prosthetic foot in accordance with the present disclosure is shown as a flow diagram. The method 1200 includes, at block 1205, providing a base spring, a connector, a heel cushion, first and second spring members, and at least one spacer. Block 1210 includes arranging the first and second spring members in parallel with each other with portions of the second spring member positioned below the first spring member. The second spring member may also be positioned rearward of the first spring member. The method 1200 includes, at block 1215, connecting distal ends of the first and second spring members to each other, such as bonding together with an elastomeric material. The first and second spring members may be spaced apart from each other at their distal and/or anterior ends while still being connected to each other. Block 1220 includes arranging the spacer between proximal ends of the first and second spring members. The spacer spaces the first and second members apart when the prosthetic foot is in a rest position. The method 1200 includes, at block 1225, connecting the proximal ends of the first and second spring members to each other and to the connector with at least one fastener. In some arrangements, the proximal ends of the first and second spring members are arranged substantially vertically. Block 1230 includes connecting the distal ends of the second spring member to a toe end portion of the base spring, such as a bond connection using an adhesive. The distal ends of the first and second spring members may be arranged substantially parallel with a top surface of the base spring. Block 1235 includes mounting the heel cushion to the base spring at a location spaced forward of a posterior end of the base spring, the heel cushion arranged to contact a bottom surface of the second spring member.

The method 1200 may also include providing the first and second spring members with a lower portion and an upper portion, arranging the lower portion at an angle of about 5° to about 30° relative to a horizontal plane, and arranging the upper portion substantially vertically. The method 1200 may include providing the base spring with a sandal slot formed in the toe end portion, and connecting the distal end of the second spring member to the base spring at a location spaced posterior of the sandal slot. The method 1200 may include moving a portion of the first spring member into contact with the second spring member or reducing the gap distance between the first and second springs during use of the prosthetic foot.

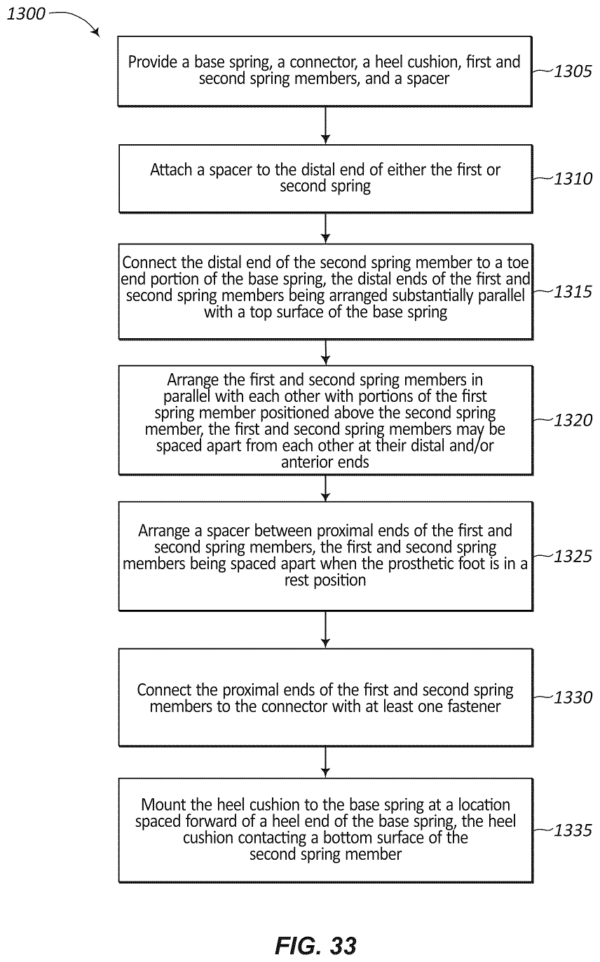

Referring now to FIG. 33, an example method 1300 of manufacturing and/or assembling a prosthetic foot in accordance with the present disclosure is shown as a flow diagram. The method 1300 includes, at block 1305, providing a base spring, a connector, a heel cushion, first and second spring members, and at least one spacer. Block 1310 includes attaching a first spacer to the distal end of either the first or second spring. Block 1315 includes connecting the distal ends of the second spring member to a toe end portion of the base spring, such as with a bond connection using an adhesive. The distal ends of the first and second spring members may be arranged substantially parallel with a top surface of the base spring. Block 1320 includes arranging the first and second spring members in parallel with each other with portions of the first spring member positioned above the second spring member. The first and second spring members may be spaced apart from each other at their distal ends and/or anterior ends. The method 1300 may also include, at block 1325, arranging a second spacer between proximal ends of the first and second spring members. The second spacer spaces the first and second members apart when the prosthetic foot is in a rest position. The method 1300 may further include, at block 1330, connecting the proximal ends of the first and second spring members to each other and to the connector with at least one fastener. In some arrangements, the proximal ends of the first and second spring members may be arranged substantially vertically. Block 1335 includes mounting the heel cushion to the base spring at a location spaced forward of a posterior end of the base spring. The heel cushion may be arranged to contact a bottom surface of the second spring member.

The method 1300 may also include providing the first and second spring members with a lower portion and an upper portion, and arranging the lower portion at an angle of about 5° to about 30° relative to a horizontal plane. The method 1300 may include providing the base spring with a sandal slot formed in the toe end portion, and connecting the distal end of the second spring member to the base spring at a location spaced posterior of the sandal slot. The method 1300 may include moving a portion of the first spring member into contact with the second spring member or reducing the gap distance between the first and second springs during use of the prosthetic foot. The method 300 may include arranging the first spring member positioned forward of the second spring member.

The methods 1200 and 1300 may be modified or altered in accordance with the present disclosure to include more or fewer steps than those illustrated in the figures. Accordingly, the flow diagrams shown in FIG. 32 and FIG. 33 may include any step or variation of methods, features and/or functionality described herein. Other types of methods are possible in accordance with the present disclosure including, for example, methods of using a prosthetic foot, methods of storing and releasing energy in a prosthetic foot during use, and methods related to adapting or customizing performance of a prosthetic foot (e.g., by interchanging a heel cushion, a top spring assembly, or connection materials, such as the first spacer 1044 or toe end connection 1092).

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. A prosthetic foot, comprising:
a base spring having a toe end portion and a heel end portion;
a top spring assembly, comprising:
a first spring member having a distal end and a proximal end;
a second spring member extending substantially parallel with and spaced apart from the first spring member along substantially an entire length of the first spring member, the second spring member having a distal end and a proximal end;
a first connection provided between the distal ends of the first and second spring members, and a second connection provided between the distal end of the second spring member and a top surface of the base spring;
a first portion arranged at a first angle relative to a horizontal plane;
a second portion extending from the first portion;
a slot extending rearward from the distal ends of the first and second spring members along at least half of a length of the first portion of the top spring assembly;
a connector connected to the proximal ends of the first and second spring members, and configured to connect the prosthetic foot to a lower limb prosthesis;
a heel cushion mounted to the base spring at a location spaced forward of a heel end of the base spring, the heel cushion arranged to contact a bottom surface of the second spring member during use of the prosthetic foot.

2. The prosthetic foot of claim 1, wherein the first angle is in the range of about 5° to about 30°.

3. The prosthetic foot of claim 1, wherein the second portion extends from the first portion in a substantially vertical direction.

4. The prosthetic foot of claim 1, further comprising a spacer positioned between the proximal ends of the first and second spring members.

5. The prosthetic foot of claim 1, wherein the first connection provides a spacing between the distal ends of the first and second spring members.

6. The prosthetic foot of claim 1, wherein the first connection is provided by an adhesive.

7. The prosthetic foot of claim 1, wherein the second spring member is spaced apart from the first spring member by spacers positioned at the distal and proximal ends of the first and second spring members.

8. The prosthetic foot of claim 1, wherein a thickness of the first spring member is within 15% of a thickness of the second spring member.

9. A prosthetic foot, comprising:
a connector configured to connect the prosthetic foot to a lower limb prosthesis;
a base spring having a toe end portion and a heel end portion;
a top spring assembly, comprising:
a first spring member having a distal end and a proximal end;
a second spring member positioned between the base spring and the first spring member, the second spring member having a distal end and a proximal end;
a first spacer positioned between the distal ends of the first and second spring members;
a second spacer positioned between the proximal ends of the first and second spring members, the first and second spacers configured to space the second spring member away from the first spring member when the prosthetic foot is in a rest state, wherein during use of the prosthetic foot, a portion of the first spring member spaced between the distal and proximal ends of the first spring member moves relative to the second spring member;
a distal end portion connected to the base spring and extending substantially horizontally;
a proximal end portion connected to the connector;
a heel cushion mounted to the base spring at a location spaced forward of a heel end of the base spring, the heel cushion arranged to contact a bottom surface of the second spring member during use of the prosthetic foot.

10. The prosthetic foot of claim 9, wherein the distal end portion of the top spring assembly is connected to the base spring with a permanent bond connection, and material of the permanent bond connection comprises an elastomeric material.

11. The prosthetic foot of claim 9, wherein the proximal end portion of the top spring assembly includes the proximal ends of the first and second spring members and is releasably connected to the connector with at least one fastener.

12. The prosthetic foot of claim 9, wherein the distal ends of the first and second spring members are connected to each other with a bond connection, and material of the bond connection defines the first spacer.

13. The prosthetic foot of claim 9, wherein the top spring assembly has a first portion arranged at an angle in the range of about 5° to about 30° relative to a horizontal plane, and a second portion extending substantially vertically.

14. The prosthetic foot of claim 9, wherein the top spring assembly is spaced apart from the base spring.

15. The prosthetic foot of claim 12, wherein the material of the bond connection comprises an elastomeric material.

16. The prosthetic foot of claim 9, wherein the heel cushion is releasably attached to the base spring, and the heel cushion comprises an elastomeric material.

17. The prosthetic foot of claim 9, wherein the distal end of the second spring member extends distally further than the distal end of the first spring member.

18. A method of manufacturing a prosthetic foot, comprising:
providing a base spring, a connector, a heel cushion, first and second spring members, and a spacer;
arranging the first and second spring members in parallel with each other with portions of the second spring member positioned below the first spring member;
connecting distal ends of the first and second spring members to each other;
arranging the spacer between proximal ends of the first and second spring members, the first and second spring members being spaced apart when the prosthetic foot is in a rest position;
connecting the proximal ends of the first and second spring members to the connector with at least one fastener;
connect the distal end of the second spring member to a toe end portion of the base spring, the distal ends of the first and second spring members being arranged substantially parallel with a top surface of the base spring;
mounting the heel cushion to the base spring at a location spaced forward of a heel end of the base spring, the heel cushion arranged to contact a bottom surface of the second spring member.

19. The method of claim 18, further comprising:
providing the first and second spring members with a lower portion and an upper portion;
arranging the lower portion at an angle of about 5° to about 30° relative to a horizontal plane;
arranging the upper portion substantially vertically.

20. The method of claim 18, wherein the base spring includes a sandal slot formed in the toe end portion, and connecting the distal end of the second spring member to the base spring includes bonding to the base spring at a location spaced posterior of the sandal slot.
